(19) United States
(12) Patent Application Publication
(10) Pub. No.: US 2023/0181648 A1
(43) Pub. Date: Jun. 15, 2023

(54) COMPOSITIONS AND METHODS FOR THE TREATMENT OR PROPHYLAXIS OF A PERFUSION DISORDER

(71) Applicant: Indiana University Research and Technology Corp, Indianapolis, IN (US)

(72) Inventors: Mervin C. Yoder, Indianapolis, IN (US); David P. Basile, Indianapolis, IN (US)

(21) Appl. No.: 17/902,482

(22) Filed: Sep. 2, 2022

Related U.S. Application Data
(62) Division of application No. 16/311,633, filed on Dec. 19, 2018, now abandoned.

(51) Int. Cl. A61K 35/44; A61P 9/10; A61P 13/12; A61K 45/06; C12N 5/071

(52) U.S. Cl. CPC A61K 35/44; A61P 9/10; A61P 13/12; A61K 45/06; C12N 5/069; C12N 2506/02; C12N 2506/03; C12N 2506/45

(57) ABSTRACT

The present disclosure provides compositions and methods for the treatment or prophylaxis of a perfusion disorder, such as ischemia and/or reperfusion injury, in a subject's organ, tissue or extremity by preserving or improving endothelial function, reducing vascular injury, and/or promoting vascular repair. The disclosed compositions comprise endothelial colony-forming cells or a serum-free composition comprising chemically defined media conditioned by endothelial colony-forming cells.

### CROSS-REFERENCE TO RELATED APPLICATIONS

This application is a divisional application of U.S. application Ser. No. 16/311,633, filed on Dec. 19, 2018, which is a U.S. National Phase filing of International Application No. PCT/US2018/0190630, filed on Feb. 21, 2018, the entire disclosure of each which are incorporated herein by reference in their entirety.

### STATEMENT REGARDING FEDERALLY SPONSORED RESEARCH OR DEVELOPMENT

This invention was made with government support under DK063114 awarded by National Institutes of Health. The government has certain rights in the invention.

### FIELD OF THE DISCLOSURE

The present disclosure pertains generally to the field of cell therapy for the treatment of perfusion disorders.

### BACKGROUND OF THE DISCLOSURE

A perfusion disorder is the process in which the delivery of oxygenated blood to tissues, organs and extremities is compromised as a result of physical trauma, systemic disease or vascular disease. The leading cause of perfusion disorders worldwide is undoubtedly atherosclerosis, a vascular disease in which plaque builds up in the arteries. The narrowing of the arteries over time limits the flow of oxygen-rich blood to the organs and other parts of your body leading to coronary artery disease, carotid artery disease, peripheral arterial disease and chronic kidney disease depending on the artery affected. As the disease progresses, the decreased blood flow can result in ischemia of downstream tissues. In addition, atherosclerotic plaque may rupture, followed rapidly by thrombotic occlusion of the vessel and death of the tissue.

Anti-thrombotic and mechanical strategies to re-open the diseased vessel reduce the duration of ischemia, leading to a prompt reperfusion of the injured myocardium. However, reperfusion itself triggers a wave of injury which together can culminate in cell death. Indeed, it is estimated that up to half of the injury of myocardial infarction stems from the reperfusion injury. Unfortunately, no clinically relevant therapies currently exist that target reperfusion injury, which means that nearly half of the injury to the heart (or brain, in the case of stroke) is not currently amenable to therapy.

For the foregoing reasons, there is an unmet, urgent need in the art for safe and effective therapies that mitigate and/or prevent ischemic and/or reperfusion injury.

### SUMMARY OF THE DISCLOSURE

Ischemia-reperfusion (I/R) events impair vascular function, reducing blood flow in tissues and organs, while promoting parenchymal cell damage and sustained tissue/organ injury. Damage to the vasculature resulting from I/R events reduces endothelial function. This damage may be permanent, since there is little evidence that endothelial cells are able to undergo a significant amount of proliferation or repair. The endothelial cell has therefore emerged as an important target in the injury process.

The present disclosure describes compositions and methods for use in treating various perfusion disorders, including ischemic and/or reperfusion injury to organs, tissues or extremities. By improving endothelial function, for example, by reducing vascular injury and by promoting vascular repair.

In one aspect, the disclosure provides a method for the treatment or prophylaxis of a perfusion disorder in a subject's organ, tissue or extremity comprising administering to the subject a composition comprising a therapeutically effective amount of endothelial colony-forming cells (ECFCs). The perfusion disorder can be caused by physical trauma or vascular disease, such as ischemia and/or reperfusion injury of the subject's organ, tissue or extremity.

In an embodiment of the first aspect, the endothelial colony-forming cells (ECFCs) are high proliferative potential ECFCs ((HPP)-ECFCs).

In an embodiment of the first aspect, the endothelial colony-forming cells (ECFCs) are derived from multipotent stem cells such as cord stem cells.

In an embodiment of the first aspect, the endothelial colony-forming cells (ECFCs) are derived from pluripotent stem cells.

In an embodiment of the first aspect, endothelial colony-forming cells (ECFCs) are derived from pluripotent stem cells without co-culture with bone marrow cells.

In an embodiment of the first aspect, the endothelial colony-forming cells (ECFCs) are derived from pluripotent stem cells without embryoid body formation.

In an embodiment of the first aspect, the endothelial colony-forming cells (ECFCs) do not express α-smooth muscle actin (α-SMA).

In an embodiment of the first aspect, the pluripotent stem cells express at least one of the transcription factors selected from the group consisting of OCT4A, NANOG, and SOX2.

In an embodiment of the first aspect, the pluripotent stem cells are embryonic stem cells, adult stem cells or induced pluripotent stem cells, e.g. induced pluripotent stem cells generated from the subject's somatic cells.

In an embodiment of the first aspect, the subject's organ or tissue is from the musculoskeletal system, circulatory system, nervous system, integumentary system, digestive system, respiratory system, immune system, urinary system, reproductive system or endocrine system.

In an embodiment of the first aspect, the organ is the subject's heart, lung, brain, liver or kidney.

In an embodiment of the first aspect, the tissue is an epithelial, connective, muscular, or nervous tissue.

In an embodiment of the first aspect, the tissue is cerebral, myocardial, lung, renal, liver, skeletal, or peripheral tissue.

In an embodiment of the first aspect, the administration of the composition comprising the endothelial colony-forming cells (ECFCs) enhances blood flow, restores endothelial cell function or promotes neovascularization in the subject's organ, tissue or extremity.

In an embodiment of the first aspect, the administration of the composition comprising the endothelial colony-forming cells (ECFCs) reduces adhesion molecule expression, such as ICAM1, or the infiltration of inflammatory cells in the subject's organ, tissue or extremity.

In an embodiment of the first aspect, the composition comprising the endothelial colony-forming cells (ECFCs) is administered directly to the subject's organ, tissue or extremity in vivo or ex vivo, after which, the organ or tissue is transplanted into the subject.

In an embodiment of the first aspect, the composition comprising the endothelial colony-forming cells (ECFCs) is administered intravenously to the subject.

In an embodiment of the first aspect, the subject has atherosclerosis, diabetes and/or cancer.

In an embodiment of the first aspect, the composition comprises endothelial colony-forming cells in a single cell suspension or disposed in a three-dimensional scaffold.

In an embodiment of the first aspect, the composition further comprises an angiogenic factor.

In a second aspect, the disclosure provides for a serum-free composition comprising a chemically defined medium conditioned by endothelial colony-forming cells.

In an embodiment of the second aspect, the endothelial colony-forming cells (ECFCs) are high proliferative potential ECFCs ((HPP)-ECFCs).

In an embodiment of the second aspect, the endothelial colony-forming cells (ECFCs) are derived from multipotent stem cells such as cord stem cells.

In an embodiment of the second aspect, the endothelial colony-forming cells (ECFCs) are derived from pluripotent stem cells.

In an embodiment of the second aspect, endothelial colony-forming cells (ECFCs) are derived from pluripotent stem cells without co-culture with bone marrow cells.

In an embodiment of the second aspect, the endothelial colony-forming cells (ECFCs) are derived from pluripotent stem cells without embryoid body formation.

In an embodiment of the second aspect, the endothelial colony-forming cells (ECFCs) do not express α-smooth muscle actin (α-SMA).

In an embodiment of the second aspect, the pluripotent stem cells express at least one of the transcription factors selected from the group consisting of OCT4A, NANOG, and SOX2.

In an embodiment of the second aspect, the pluripotent stem cells are embryonic stem cells, adult stem cells or induced pluripotent stem cells, e.g. induced pluripotent stem cells generated from the subject's somatic cells.

In a third aspect, the present disclosure provides for a method for the treatment or prophylaxis of a perfusion disorder in a subject's organ, tissue or extremity comprising administering to the subject a therapeutically effective amount of a serum-free composition comprising a chemically defined medium conditioned by endothelial colony-forming cells (ECFCs). The perfusion disorder can be caused by physical trauma or vascular disease, such as ischemia and/or reperfusion injury of the subject's organ, tissue or extremity.

In an embodiment of the third aspect, the endothelial colony-forming cells (ECFCs) are high proliferative potential ECFC ((HPP)-ECFC).

In an embodiment of the third aspect, the endothelial colony-forming cells (ECFCs) are derived from multipotent stem cells such as cord stem cells.

In an embodiment of the third aspect, the endothelial colony-forming cells (ECFCs) are derived from pluripotent stem cells.

In an embodiment of the third aspect, endothelial colony-forming cells (ECFCs) are derived from pluripotent stem cells without co-culture with bone marrow cells.

In an embodiment of the third aspect, the endothelial colony-forming cells (ECFCs) are derived from pluripotent stem cells without embryoid body formation.

In an embodiment of the third aspect, the endothelial colony-forming cells (ECFCs) do not express α-smooth muscle actin (α-SMA).

In an embodiment of the third aspect, the pluripotent stem cells express at least one of the transcription factors selected from the group consisting of OCT4A, NANOG, and SOX2.

In an embodiment of the third aspect, the pluripotent stem cells are embryonic stem cells, adult stem cells or induced pluripotent stem cells, e.g. induced pluripotent stem cells generated from the subject's somatic cells.

In an embodiment of the third aspect, the subject's organ or tissue is from the musculoskeletal system, circulatory system, nervous system, integumentary system, digestive system, respiratory system, immune system, urinary system, reproductive system or endocrine system.

In an embodiment of the third aspect, the organ is the subject's heart, lung, brain, liver or kidney.

In an embodiment of the third aspect, the tissue is an epithelial, connective, muscular, or nervous tissue.

In an embodiment of the third aspect, the tissue is cerebral, myocardial, lung, renal, liver, skeletal, or peripheral tissue.

In an embodiment of the third aspect, the administration of the composition comprising the endothelial colony-forming cells (ECFCs) enhances blood flow, restores endothelial cell function or promotes neovascularization in the subject's organ, tissue or extremity.

In an embodiment of the third aspect, the administration of the composition comprising the endothelial colony-forming cells (ECFCs) reduces adhesion molecule expression or the infiltration of inflammatory cells in the subject's organ, tissue or extremity.

In an embodiment of the third aspect, the composition comprising the endothelial colony-forming cells (ECFCs) is administered directly to the subject's organ, tissue or extremity in vivo or ex vivo, after which, the organ or tissue is transplanted into the subject.

In an embodiment of the third aspect, the composition comprising the endothelial colony-forming cells (ECFCs) is administered intravenously to the subject.

In an embodiment of the third aspect, the subject has atherosclerosis, diabetes and/or cancer.

In an embodiment of the third aspect, the composition comprises endothelial colony-forming cells in a single cell suspension or disposed in a three-dimensional scaffold.

In an embodiment of the third aspect, the composition further comprises an angiogenic factor.

In a fourth aspect, the disclosure provides for a kit comprising a serum-free composition comprising a chemically defined medium conditioned by endothelial colony-forming cells (ECFCs).

Other features and advantages of the disclosure will be apparent from the following detailed description and from the Exemplary Embodiments.

### BRIEF DESCRIPTION OF THE DRAWINGS

These and other features of the disclosure will become more apparent in the following detailed description in which reference is made to the appended drawings wherein:

FIGS. 1A-1E provide an exemplary depiction of the functional and structural recovery of the kidney following the administration of rat pulmonary microvascular endothelial cells (PMVEC). Data in FIGS. 1A, 1C and 1E are presented as means±SE. * and # indicate P<0.05 in PMVEC-treated rats compared with pulmonary artery endothelial cells (PAEC)-treated and vehicle-treated rats, respectively, by Student's t-test.

FIG. 1A is an exemplary graph showing serum creatinine (sCre) levels for 7 days following FR or sham surgery (n=3) in rats treated with vehicle (n=7), PAEC (n=6), or PMVEC (n=8).

FIG. 1B shows representative microscopic images of periodic acid-Schiff (PAS)-stained kidney sections following 7 days of recovery from renal FR.

FIG. 1C is an exemplary graph showing sCre levels for 2 days following I/R or sham surgery in vehicle-treated (n=6) vs. PMVEC-treated (n=6) rats.

FIG. 1D shows representative microscopic images of PAS-stained kidney sections following 2 days of recovery from renal I/R.

FIG. 1E is an exemplary graph showing the tissue injury score in renal tissues from 2-day post-ischemic rats.

FIGS. 2A-2B show an example of rat PMVEC preserve medullary blood flow in the early post-ischemic period. Data are averaged in 10-min time bins normalized to the baseline values for each rat. Data are presented as means±SE. * indicates P<0.05 in PMVEC-treated rats compared with vehicle-treated rats by ANOVA with repeated measures.

FIG. 2A is an exemplary graph showing total renal blood flow measured for 30 min before ischemia and up to 120 min post-reperfusion.

FIG. 2B is an exemplary graph showing medullary blood flow measured for 30 min before ischemia and up to 120 min post-reperfusion.

FIGS. 3A-3D are representative confocal microscopic images showing that rat PMVEC do not home to the kidney following transplantation.

FIG. 3A depicts a representative confocal microscopic image of freshly suspended PMVEC fluorescently labeled with cell tracker red in vitro and imaged before transplantation.

FIG. 3B depicts a representative confocal microscopic image of kidney tissue section imaged 2 h post-transplantation.

FIG. 3C depicts a representative confocal microscopic image of a kidney tissue section imaged 2 days post-transplantation.

FIG. 3D depicts a representative confocal microscopic image of spleen tissue section, showing fluorescently labeled cells with a similar size and fluorescence intensity of pre-infused PMVEC (white arrows).

FIGS. 4A-4D show an example of human endothelial colony-forming cells-conditioned medium (ECFC-CM) protecting against renal I/R injury. Data in FIGS. 4A, C and D are presented as means±SE. * indicates P<0.05 in ECFC-CM-treated compared with vehicle-treated rats by Student's t-test. n.d., not detectable.

FIG. 4A is an exemplary graph showing serum creatinine (sCre) levels for 2 days following I/R or sham surgery (n=3) in vehicle-treated (n=7) and ECFC-CM-treated rats (n=7).

FIG. 4B shows a representative microscopic images of PAS-stained rat kidney sections following 2 days of recovery from renal I/R.

FIG. 4C is an exemplary graph showing the tissue injury score in renal tissues from 2-day post-ischemic rats.

FIG. 4D is an exemplary graph showing KIM-1 mRNA expression in sham-treated, vehicle-treated, or ECFC-CM-treated rats.

FIGS. 5A-5B show an example of human ECFC-CM preserving medullary blood flow in the early post-ischemic period. Data are averaged in 10-min time bins normalized to the baseline values for each rat. Data are presented as means±SE. * indicates P<0.05 in ECFC-CM-treated rats compared with vehicle-treated rats by ANOVA with repeated measures.

FIG. 5A is an exemplary graph showing total renal blood flow measured for 30 min before ischemia and up to 120 min post-reperfusion.

FIG. 5B is an exemplary graph showing medullary blood flow measured for 30 min before ischemia and up to 120 min post-reperfusion.

FIGS. 6A-6C show an example of human ECFC-CM reducing adhesion molecule expression following recovery from I/R injury. In FIGS. 6A and 6C * indicates P<0.05 in FR+vehicle-treated rats compared to sham-operated rats by Student's t-test. # indicates P<0.05 in I/R+ECFC-CM-treated rats compared to I/R+vehicle-treated rats by Student's t-test. n.d., not detectable.

FIG. 6A is an exemplary graph showing ICAM-1 mRNA expression levels in samples derived from whole kidney using real-time PCR. Rats were treated with vehicle or ECFC-CM as labeled and subjected to sham surgery or renal I/R, followed by 5 h recovery.

FIG. 6B shows representative microscopic images of ICAM-1 immunofluorescence in kidney sections from sham, vehicle-treated, or ECFC-CM-treated rats.

FIG. 6C is an exemplary graph depicting the fraction of the total area occupied by ICAM-1 immunofluorescent stained structures. Immunofluorescence data are presented as % of total area compared with the mean value of sham-operated control rats.

FIGS. 7A-7G show an example of human ECFC-CM reducing infiltration of inflammatory cells in kidneys following FR. Kidney resident monocytes were isolated from rat kidneys harvested 2 days post-surgery/treatment. Data in FIGS. 7B-7G are presented as means±SE. * indicates P<0.05 in I/R+vehicle-treated rats compared to sham-operated rats by Student's t-test. Φ indicates P<0.05 in I/R+ECFC-CM-treated rats compared to sham-operated rats by Student's t-test. # indicates P<0.05 in FR+ECFC-CM-treated rats compared to FR+vehicle-treated rats by Student's t-test.

FIG. 7A is an exemplary schematic depicting the gating strategy for fluorescence-activated cell sorting (FACS) analysis. Lymphocytes were gated based on the Forward Scatter vs. Side Scatter plot.

FIG. 7B is an exemplary graph showing the number of infiltrating monocytes per gram of kidney tissue harvested from sham, vehicle-treated, or ECFC-CM-treated rats.

FIG. 7C is an exemplary graph showing the number of CD4+ T cells per gram of kidney tissue in the samples described in FIG. 7B.

FIG. 7D is an exemplary graph showing the number of CD8+ T cells per gram of kidney tissue in the samples described in FIG. 7B.

FIG. 7E is an exemplary graph showing the number of IL-17+ T cells per gram of kidney tissue in the samples described in FIG. 7B.

FIG. 7F is an exemplary graph showing the number of CD4+IL-17+ T cells per gram of kidney tissue in the samples described in FIG. 7B.

FIG. 7G is an exemplary graph showing the number of CD4+IFN-γ+ T cells per gram of kidney tissue in the samples described in FIG. 7B.

### DETAILED DESCRIPTION

Compositions and methods are disclosed for use in treating perfusion disorders affecting tissues, organs or extremities. That the disclosure may be more readily understood, select terms are defined below.

### Definitions

The terminology used herein is for the purpose of describing particular embodiments only and is not intended to be limiting of example embodiments of the invention. Unless defined otherwise, all technical and scientific terms used herein generally have the same meaning as commonly understood by one of ordinary skill in the art to which this disclosure belongs.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

A “subject” is a vertebrate, preferably a mammal (e.g., a non-human mammal), more preferably a primate and still more preferably a human. Mammals include, but are not limited to, primates, humans, farm animals, sport animals, and pets.

Perfusion is the process by which a fluid passes through the circulatory system or lymphatic system of an organ, tissue, or extremity, e.g. the delivery of blood to a capillary bed in a tissue.

As used herein, a “perfusion disorder” or “perfusion disease” is any pathological process that deprives a subject's tissue, organ or extremity of oxygenated blood. A perfusion disorder can be caused by physical trauma or as a consequence of systemic or vascular disease that reduces arterial flow to an organ, tissue of extremity. Physical trauma can include, for example, a chronic obstructive process, or injury resulting from a physical insult such as frostbite or radiation.

As used herein, a “vascular disease” refers to a disease of the vessels, primarily arteries and veins, which transport blood to and from the heart, brain and peripheral organs such as, without limitation, the arms, legs, kidneys and liver. In particular “vascular disease” refers to the coronary arterial and venous systems, the carotid arterial and venous systems, the aortic arterial and venous systems and the peripheral arterial and venous systems. The disease that may be treated is any that is amenable to treatment with the compositions disclosed herein, either as the sole treatment protocol or as an adjunct to other procedures such as surgical intervention. The disease may be, without limitation, atherosclerosis, vulnerable plaque, restenosis, peripheral arterial disease (PAD) or critical limb ischemia (CLI). Peripheral vascular disease includes arterial and venous diseases of the renal, iliac, femoral, popliteal, tibial and other vascular regions.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that occlude the lumen of the artery, a process called stenosis. When the stenosis becomes severe enough, the blood supply to the organ supplied by the particular artery is depleted resulting in a stroke, if the afflicted artery is a carotid artery, heart attack if the artery is coronary, or loss of organ or limb function if the artery is peripheral.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet. In particular at present a peripheral vascular disease often refers to a vascular disease of the superficial femoral artery.

“Critical limb ischemia” (CLI) is an advanced stage of peripheral artery disease (PAD). It is defined as a triad of ischemic rest pain, arterial insufficiency ulcers, and gangrene. The latter two conditions are jointly referred to as tissue loss, reflecting the development of surface damage to the limb tissue due to the most severe stage of ischemia. Over 500,000 patients in the U.S. each year are diagnosed with critical limb ischemia (CLI). Half the patients die from a cardiovascular cause within 5 years, a rate that is 5 times higher than a matched population without CLI (Varu et al. (2010) Journal of Vascular Surgery 51(1): 230-41; Rundback et al. Ann. Vasc. Surg. (2017) 38:191-205).

“Restenosis” refers to the re-narrowing of an artery at or near the site where angioplasty or another surgical procedure was previously performed to remove a stenosis. It is generally due to smooth muscle cell proliferation and, at times, is accompanied by thrombosis.

“Vulnerable plaque” refers to an atheromatous plaque that has the potential of causing a thrombotic event and is usually characterized by a thin fibrous cap separating a lipid filled atheroma from the lumen of an artery. The thinness of the cap renders the plaque susceptible to rupture. When the plaque ruptures, the inner core of usually lipid-rich plaque is exposed to blood. This releases tissue factor and lipid components with the potential of causing a potentially fatal thrombotic event through adhesion and activation of platelets and plasma proteins to components of the exposed plaque.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a perfusion disorder or disease, e.g. an ischemia-reperfusion (I/R) injury. The term “treating” includes reducing or alleviating at least one adverse effect or symptom of a condition, disease or disorder associated with a perfusion disorder. Treatment is generally “effective” if one or more symptoms or clinical markers are reduced. Alternatively, treatment is “effective” if the progression of a perfusion disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a composition as disclosed herein into a subject by a method or route which results in at least partial delivery of the composition at a desired site. Pharmaceutical compositions disclosed herein can be administered by any appropriate route which results in an effective treatment in the subject.

In one embodiment, an “effective amount” refers to the optimal number of cells needed to elicit a clinically significant improvement in the symptoms and/or pathological state associated with a perfusion disorder including slowing, stopping or reversing cell death, reducing a neurological deficit or improving a neurological response. The therapeutically effective amount can vary depending upon the intended application or the subject and disease condition being treated, e.g., the weight and age of the subject, the severity of the disease condition, the manner of administration and the like, which can readily be determined by one of ordinary skill in the art, e.g., a board-certified physician.

As used herein, “primary endothelial cells” refers to endothelial cells found in the blood, and which display the potential to proliferate and form an endothelial colony from a single cell and have a capacity to form blood vessels in vivo in the absence of co-implanted or co-cultured cells.

As used herein, “endothelial colony-forming cells” and “ECFCs” refer to non-primary endothelial cells that are generated in vitro, e.g. from human pluripotent stem cells (hPSCs). ECFCs have various characteristics, at least including the potential to proliferate and form an endothelial colony from a single cell and have a capacity to form blood vessels in vivo in the absence of co-implanted or co-cultured cells. In an embodiment, ECFCs have the following characteristics: (A) characteristic ECFC molecular phenotype; (B) capacity to form capillary-like networks in vitro on Matrigel™; (C) high proliferation potential; (D) self-replenishing potential; (E) capacity for blood vessel formation in vivo without co-culture with any other cells; (F) increased cell viability and/or decreased senescence and (G) cobblestone morphology.

In certain embodiment, the ECFCs or ECFC-like cells express one or more markers chosen from CD31, NRP-1, CD144 and KDR. In one embodiment, the ECFCs express two or more markers chosen from CD31, NRP-1, CD144 and KDR. In one embodiment, the ECFCs express three or more markers chosen from CD31, NRP-1, CD144 and KDR. In one embodiment, the ECFCs express four or more markers chosen from CD31, NRP-1, CD144 and KDR.

As used herein, “endothelial colony-forming like cells” and “ECFC-like cells” refer to non-primary endothelial cells that are generated in vitro from an endothelial progenitor or endothelial progenitor cells, KDR⁺NCAM⁺APLNR⁺ mesoderm (MSD) cells. ECFC-like cells have various characteristics, at least including the potential to proliferate and form an endothelial colony from a single cell and have a capacity to form blood vessels in vivo in the absence of co-implanted or co-cultured cells. In an embodiment, ECFC-like cells have properties similar to ECFCs including (A) characteristic ECFC molecular phenotype; (B) capacity to form capillary-like networks in vitro on Matrigel™; (C) high proliferation potential; (D) self-replenishing potential; (E) capacity for blood vessel formation in vivo without co-culture with any other cells; (F) increased cell viability and/or decreased senescence and (G) cobblestone morphology.

As used herein, the terms “high proliferation potential”, “high proliferative potential” and “HPP” refer to the capacity of a single cell to divide into more than about 2000 cells in a 14-day cell culture. Preferably, HPP cells have a capacity to self-replenish. For example, the HPP-ECFCs provided herein have a capacity to self-replenish, meaning that an HPP-ECFC can give rise to one or more HPP cells within a secondary HPP-ECFC colony when replated in vitro.

Various techniques for measuring proliferative potential of cells are known in the art and can be used with the methods provided herein to confirm the proliferative potential of the ECFC. For example, single cell assays such as those described in PCT publication WO 2015/138634 may be used to evaluate the clonogenic proliferative potential of ECFC. In general, an ECFC to be tested for proliferative potential may be treated to obtain a single cell suspension. The suspended cells are counted, diluted and single cells are cultured in each well of 96-well plates. After several days of culture, each well is examined to quantitate the number of cells. Those wells containing two or more cells are identified as positive for proliferation. Wells with ECFC counts of 1 are categorized as non-dividing, wells with ECFC counts of 2-50 are categorized as endothelial cell clusters (ECC), wells with ECFC counts of 51-500 or 501-2000 are categorized as low proliferative potential (LPP) cells and wells with ECFC counts of 2001 or greater are categorized as high proliferative potential (HPP) cells.

As used herein, “cord blood ECFCs” and “CB-ECFCs” refer to ECFCs that are derived from umbilical cord blood.

The term “pluripotent” or “pluripotency” refers to cells with the ability to give rise to progeny that can undergo differentiation, under the appropriate conditions, into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal. A standard art-accepted test, such as the ability to form a teratoma in 8-12-week-old SCID mice, can be used to establish the pluripotency of a cell population, however identification of various pluripotent stem cell characteristics can also be used to detect pluripotent cells.

Pluripotent stem cell characteristics refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. Embryonic stem cells, primordial germ cells (EGCs) and iPSCs are considered to be pluripotent.

“Multipotent cells” can develop into more than one cell type but are more limited than pluripotent cells. Adult stem cells such as hematopoietic stem cells and cord blood stem cells are considered multipotent.

As used herein, “induced pluripotent stem cells,” “IPS cells” or “iPSC” refer to a type of pluripotent stem cell that has been generated from a non-pluripotent cell, such as, for example, an adult somatic cell, or a terminally differentiated cell, such as, for example, a fibroblast, a hematopoietic cell, a myocyte, a neuron, an epidermal cell, or the like, by introducing into the non-pluripotent cell or contacting the non-pluripotent cell with a specific combination of stem cell transcription factors (e.g. Oct-3/4, Sox2, KLF4 and c-Myc; see, Takahashi, K. & Yamanaka, S. Cell 126, 663-676 (2006); Okita, K. et al. Nature 448, 313-317 (2007); Wernig, M. et al. Nature 448, 318-324 (2007); Maherali, N. et al. Cell Stem Cell 1, 55-70 (2007); Meissner et al. Nature Biotechnol. 25, 1177-1181 (2007); Yu, J. et al. Science 318, 1917-1920 (2007); Nakagawa, M. et al. Nature Biotechnol. 26, 101-106 (2007); Wernig et al. Cell Stem Cell 2, 10-12 (2008). In certain embodiments, iPS cells can be chemically induced from adult somatic cells (see, e.g. U.S. Pat. No. 9,394,524, the content of which is incorporated herein in its entirety).

As used herein, “adhesion molecules” whose expression is associated with ischemia/reperfusion injury include, but are not limited to, intercellular cellular adhesion molecules-1 (ICAM-1), vascular cellular adhesion molecules-1 (VCAM-1), Platelet endothelial cell adhesion molecule (PECAM-1), E-selectin, P-Selectin and the β2-integrins, LFA-1 (CD11a/CD18) and Mac-1 (CD11b/CD18).

### Methods of Generating Endothelial Colony-Forming Cells (ECFCs)

As described herein, the inventors have provided compositions comprising endothelial colony-forming cells (ECFCs) and related reagents, including compositions comprising conditioned medium obtained from ECFCs, as well as methods of using such compositions and related reagents therapeutically.

### Differentiating Cord Blood (CB) Stem Cells into Endothelial Colony Forming Cells (ECFCs).

ECFCs can be derived from human umbilical cord blood according to methods described, for example, by Yoder et al. (Yoder M C et al. Blood 109: 1801-1809, 2007). In this method, peripheral blood samples or umbilical cord blood samples are collected in citrate phosphate dextrose (CPD) solution. Human mononuclear cells (MNC) from these blood samples are diluted 1:1 with Hanks balanced salt solution (HBSS) and overlaid onto an equivalent volume of Histopaque 1077. Cells are centrifuged for 30 minutes at room temperature at 740 g. MNCs are isolated and washed 3 times with EBM-2 medium supplemented with 10% fetal bovine serum (FBS), 2% penicillin/streptomycin, and 0.25 μg/mL amphotericin B (complete EGM-2 medium). MNCs are resuspended in 12 mL complete EGM-2 medium. Cells are seeded onto 3 separate wells of a 6-well tissue culture plate pre-coated with type 1 rat tail collagen at 37° C., 5% CO₂, in a humidified incubator. After 24 hours of culture, nonadherent cells and debris are aspirated, adherent cells are washed once with complete EGM-2 medium, and complete EGM-2 medium is added to each well. Medium is changed daily for 7 days and then every other day until the first passage. Colonies of endothelial cells appear between 5 and 22 days of culture and are identified as well-circumscribed monolayers of cobblestone-appearing cells. The cells are released from the original tissue culture plates, resuspended in complete EGM-2 media, and plated onto 75-cm² tissue culture flasks coated with type 1 rat tail collagen for further passage.

### Differentiating Pluripotent Cells into Endothelial Colony Forming Cells (ECFCs).

Methods for differentiating pluripotent cells into ECFCs are known in the art and are described, for example, in PCT publication WO 2015/138634, where methods for differentiating pluripotent cells into “endothelial colony-forming cell-like cells” are described and where the “endothelial colony-forming cell-like cells” are the same as the ECFCs described.

For example, the ECFCs can be prepared by providing pluripotent stem cells, inducing them to differentiate into cells of the endothelial lineage and isolating the ECFCs from the differentiated cells of the endothelial lineage as described in PCT publication WO 2015/138634, the content of which is hereby incorporated herein in its entirety.

In certain embodiments, ECFCs are generated from one of the following cell lines: human embryonic stem cell (hESC) line H9; fibroblast-derived human iPS cell line DF19-9-11T; hiPS cell line FCB-iPS-1; or hiPS cell line FCB-iPS-2, as described, for example, in PCT publication WO 2015/138634. Alternatively, iPS cell lines are available from the ATCC, California Institute for Regenerative Medicine (CIRM) or European Bank for Induced Pluripotent Stem Cells as well as from commercial vendors.

Methods for generating an isolated population of ECFCs in vitro from pluripotent cells are known in the art. Pluripotent cells suitable for use in the methods of the present disclosure can be, for example, an embryonic stem (ES) cell, primordial germ cell or induced pluripotent stem cell.

In one embodiment, pluripotent cells are cultured under conditions suitable for maintaining pluripotent cells in an undifferentiated state. Methods for maintaining pluripotent cells in vitro, i.e., in an undifferentiated state, are well known in the art. In certain embodiments, hES and hiPS cells may be maintained in mTeSR1 complete medium on Matrigel™ in 10 cm² tissue culture dishes at 37° C. and 5% CO₂ for about two days.

Additional and/or alternative methods for culturing and/or maintaining pluripotent cells may be used. For example, as the basal culture medium, any of TeSR, mTeSR1 αMEM, BME, BGJb, CMRL 1066, DMEM, Eagle MEM, Fischer's media, Glasgow MEM, Ham, IMDM, Improved MEM Zinc Option, Medium 199 and RPMI 1640, or combinations thereof, may be used for culturing and or maintaining pluripotent cells.

The pluripotent cell culture medium used may contain serum or it may be serum- free. Serum-free refers to a medium comprising no unprocessed or unpurified serum. Serum-free media can include purified blood-derived components or animal tissue-derived components, such as, for example, growth factors. The pluripotent cell medium used may contain one or more alternatives to serum, such as, for example, knockout Serum Replacement (KSR), chemically-defined lipid concentrated (Gibco) or glutamax (Gibco).

Methods for passaging pluripotent cells are well known in the art. For example, after pluripotent cells are plated, medium may be changed on days 2, 3, and 4 and cells are passaged on day 5. Generally, once a culture container is 70-100% confluent, the cell mass in the container is split into aggregated cells or single cells by any method suitable for dissociation and the aggregated or single cells are transferred into new culture containers. Cell “passaging” is a well-known technique for keeping cells alive and growing cells in vitro for extended periods of time.

In vitro pluripotent cells can be induced to undergo endothelial differentiation. Various methods, including culture conditions, for inducing differentiation of pluripotent cells into cells of the endothelial lineage are well known in the art (e.g., see the published U.S. Patent Application No. 2017/0022476, the content of which is hereby incorporated herein in its entirety).

In one embodiment, it is preferable to induce differentiation of pluripotent cells in a chemically defined medium. For example, Stemline II serum-free hematopoietic expansion medium can be used as a basal endothelial differentiation medium supplemented with various growth factors to promote differentiation of the pluripotent cells into cells of the endothelial lineage, including ECFCs. In certain embodiments, activin A, vascular endothelial growth factor (VEGF), basic fibroblast growth factor (FGF-2) and bone morphogenetic protein 4 (BMP-4) may be added to the chemically defined differentiation medium to induce differentiation of pluripotent cells into cells of the endothelial lineage, including ECFCs.

After 2 days (-D2) of culture in a basal culture medium (e.g., mTeSR1), differentiation of pluripotent cells may be directed toward the endothelial lineage by contacting the cells for 24 hours with an endothelial differentiation medium comprising an effective amount of activin A, BMP-4, VEGF and FGF-2. Following 24 hours of differentiation, activin A is removed from the culture by replacing the medium with an endothelial differentiation medium comprising an effective amount of BMP-4, VEGF and FGF-2. By “effective amount”, is meant an amount effective to promote differentiation of pluripotent cells into cells of the endothelial lineage, including ECFCs. The endothelial differentiation medium comprising an effective amount of BMP-4, VEGF and FGF-2 may be replenished every 1-2 days.

Activin A is a member of the TGF-β superfamily that is known to activate cell differentiation via multiple pathways. Activin A facilitates activation of mesodermal specification but is not critical for endothelial specification and subsequent endothelial cell proliferation. In one embodiment, the endothelial differentiation medium comprises activin A at a concentration of about 5-25 ng/mL In one preferred embodiment, the endothelial differentiation medium comprises Activin A at a concentration of about 10 ng/mL

Bone morphogenetic protein-4 (BMP-4) is a ventral mesoderm inducer that is expressed in adult human bone marrow (BM) and is involved in modulating proliferative and differentiative potential of hematopoietic progenitor cells (Bhardwaj et al. Nat Immunol. (2001) 2(2):172-80; Bhatia et al. J Exp Med. (1999) 189(7):1139-48; Chadwick et al. Blood (2003) 102(3):906-15). Additionally, BMP-4 can modulate early hematopoietic cell development in human fetal, neonatal, and adult hematopoietic progenitor cells (Davidson and Zon, Curr Top Dev Biol. (2000) 50:45-60; Huber et al., Blood (1998) 92(11):4128-37; Marshall et al., Blood (2000) 96(4):1591-3). In one embodiment, the endothelial differentiation medium comprises BMP-4 at a concentration of about 5-25 ng/mL In one preferred embodiment, the endothelial differentiation medium comprises BMP-4 at a concentration of about 10 ng/mL.

Vascular endothelial growth factor (VEGF) is a signaling protein involved in embryonic circulatory system formation and angiogenesis. In vitro, VEGF can stimulate endothelial cell mitogenesis and cell migration. In one embodiment, the endothelial differentiation medium comprises VEGF in a concentration of about 5-50 ng/mL In one preferred embodiment, the endothelial differentiation medium comprises VEGF at a concentration of about 10 ng/mL In one particularly preferred embodiment, the endothelial differentiation medium comprises VEGF at a concentration of about 10 ng/mL

Basic fibroblast growth factor, also referred to as bFGF or FGF-2, has been implicated in diverse biological processes, including limb and nervous system development, wound healing, and tumor growth. bFGF has been used to support feeder-independent growth of human embryonic stem cells. In one embodiment, the endothelial differentiation medium comprises FGF-2 at a concentration of about 5-25 ng/mL. In one preferred embodiment, the endothelial differentiation medium comprises FGF-2 at a concentration of about 10 ng/mL.

In an embodiment, the method for generating ECFCs does not require co-culture with supportive cells, such as, for example, OP9 stromal cells. In another embodiment the method for generating ECFCs does not require embryoid body (EB) formation. In another embodiment the method for generating ECFCs does not require exogenous TGF-β inhibition.

### Differentiating ECFC Progenitor Mesoderm (MSD) Cells into ECFC-Like Cells

In certain embodiments, the present disclosure also provides a method for generating an isolated population of human KDR⁺NCAM⁺APLNR⁺ mesoderm (MSD) cells from human pluripotent stem cells. The method comprises providing pluripotent stem cells (PSCs); inducing the pluripotent stem cells to undergo mesodermal differentiation, wherein the mesodermal induction comprises: i) culturing the pluripotent stem cells for about 24 hours in a mesoderm differentiation medium comprising Activin A, BMP-4, VEGF and FGF-2; and ii) replacing the medium of step i) with a mesoderm differentiation medium comprising BMP-4, VEGF and FGF-2 about every 24-48 hours thereafter for about 72 hours; and isolating from the cells induced to undergo mesoderm differentiation, wherein their isolation comprises: iii) sorting the cells to select for KDR⁺NCAM⁺APLNR⁺ mesoderm cells (see International Application No.: PCT/US2017/045496, the content of which is incorporated by reference herein in its entirety). In certain embodiments, the sorting further comprises selection of SSEAS KDR⁺NCAM⁺APLNR⁺ cells.

In further embodiments, the isolated mesoderm cells are induced to undergo endothelial differentiation according to methods well known in the art. For example, KDR⁺NCAM⁺APLNR⁺ mesoderm MSD cells can be cultured in a chemically defined medium, e.g. Stemline II serum-free hematopoietic expansion medium, supplemented with growth factors, e.g. VEGF, FGF-2 and BMP-4. After 10-12 days in culture, the MSD cells undergo endothelial differentiation. CD31⁺CD144⁺NRP-1⁺ ECFC-like cells can then be isolated using flow cytometry.

ECFC-like cells have many of the properties of ECFCs including a cobblestone morphology and the capacity, after implantation, to form blood vessels in vivo. Importantly, as with ECFCs, the methods of generating ECFC-like cells described herein do not require co-culture with supportive cells, such as, for example, OP9 bone marrow stromal cells, embryoid body (EB) formation or exogenous TGF-β inhibition.

### Isolating ECFCs from Primary Endothelial Cells

CD3I⁺NRP-1⁺ cells can also be selected and isolated from the population of primary cells undergoing endothelial differentiation. Methods, for selecting cells having one or more specific molecular markers are well known in the art. For example, the cells may be selected based on the expression of specific cell surface markers by flow cytometry, including fluorescence-activated cell sorting, or magnetic-activated cell sorting.

In one embodiment, CD31⁺NRP-1⁺ cells can be selected from a population of cells undergoing endothelial differentiation, as described herein, on day 10, 11 or 12 of differentiation. In one preferred embodiment, CD31⁺NRP-1⁺ cells can be selected from the population of cells undergoing endothelial differentiation on day 12 of differentiation. This cell population contains a higher percentage of NRP-1⁺ cells relative to cell populations at an earlier stage of differentiation.

Adherent endothelial cells (ECs) may be harvested as a single cell suspension after day 12 of differentiation. Cells are counted and CD31⁺CD144⁺NRP-1+ cells can then be selected using flow cytometry.

The isolated CD31⁺NRP-1⁺ ECFCs can be expanded in vitro using culture conditions known in the art. In one embodiment, culture dishes are coated with type 1 collagen as a matrix attachment for the cells. Alternatively, fibronectin, Matrigel or other cell matrices may also be used to facilitate attachment of cells to the culture dish. In one embodiment, discussed further below, Endothelial Growth Medium 2 (EGM2) plus VEGF, IGF1, EGF, and FGF2, vitamin C, hydrocortisone, and fetal calf serum may be used to expand the isolated CD31⁺NRP-1⁺ ECFC cells.

CD31⁺NRP-1⁺ isolated ECFCs may be centrifuged and re-suspended in 1:1 endothelial growth medium and endothelial differentiation medium. About 2500 selected cells per well are then seeded on collagen-coated 12-well plates. After 2 days, the culture medium is replaced with a 3:1 ratio of endothelial growth medium and endothelial differentiation medium. ECFC-like colonies appear as tightly adherent cells and exhibited cobblestone morphology on day 7 of expansion.

ECFC clusters may be cloned to isolate substantially pure populations of HPP-ECFCs. In this disclosure, the term “pure” or “substantially pure” refers to a population of cells wherein at least about 75%, 85%, 90%, 95%, 98%, 99% or more of the cells are HPP-ECFCs. In other embodiments, the term “substantially pure” refers to a population of ECFCs that contains fewer than about 25%, 20%, about 10%, or about 5% of non-ECFCs.

In certain embodiments, confluent ECFCs may be passaged by plating 10,000 cells per cm2 as a seeding density and maintaining ECFCs in complete endothelial growth media (collagen coated plates and cEGM-2 media) with media change every other day.

In certain embodiments, the ECFCs generated using the methods described herein can be expanded in a composition comprising endothelium growth medium and passaged up to 18 times, while maintaining a stable ECFC phenotype. By “stable ECFC phenotype”, is meant cells exhibiting cobblestone morphology, expressing the cell surface antigens CD31 and CD144, and having a capacity to form blood vessels in vivo in the absence of co-culture and/or co-implanted cells. In a preferred embodiment, ECFCs having a stable phenotype also express CD144 and KDR but do not express α-SMA (alpha-smooth muscle actin).

In an embodiment, the method for isolating ECFCs from primary endothelial cell population does not require co-culture with supportive cells, such as, for example, OP9 stromal cells. In another embodiment the method for isolating ECFCs from primary endothelial cell population does not require embryoid body (EB) formation. In another embodiment the method isolating ECFCs from primary endothelial cell population does not require exogenous TGF-β inhibition.

### Characteristics of Isolated ECFC and ECFC-like Populations

The substantially pure human cell populations of ECFCs and ECFC-like cells described herein exhibit the following characteristics: (1) a cobblestone morphology, (2) a capacity to form capillary-like networks on Matrigel™-coated dishes, (3) a capacity to form blood vessels in vivo in the absence of co-culture and/or co-implanted cells, (4) express the cell surface markers CD31⁺CD144⁺NRP-1⁺ (5) do not express α-SMA (6) have an increased cell viability and/or decreased senescence, (7) capable of self-renewal and (8) have a high clonal proliferation potential (equal to or greater than cord blood derived ECFCs (CB-ECFCs)).

Unlike with ECFCs, ECs produced in vitro from hPSC using protocols that require co-culture with OP9 cells or EB development often express α-SMA.

In certain embodiments, about 95% or more of isolated single ECFCs proliferate and at least about 35-50% of the isolated single ECFCs are HPP-ECFCs that are capable of self-renewal.

In certain embodiments, the ECFCs and ECFC-like cells in the population comprise HPP-ECFCs having a proliferative potential to generate at least 1 trillion ECFCs ECFC-like cells from a single starting pluripotent cell.

Methods of measuring molecular expression patterns in ECs, including ECFCs and ECFC-like cells, are known in the art. For example, various known immunocytochemistry techniques for assessing expression of various markers in cells generated using the methods described can be found, for example, in PCT publication WO 2015/138634, the content of which is incorporated herein in its entirety.

The ability of ECFCs or ECFC-like cells cultured in vitro on Matrigel™ to form capillary-like networks can be evaluated using methods disclosed in PCT publication WO 2015/138634.

Endothelial cells (ECFCs) derived from hPSCs in vitro or ECFC-like cells as disclosed herein have different proliferation potentials relative to CB-ECFCs. For example, approximately 45% of single cell CB-ECFC have low proliferative potential (LPP) and approximately 37% of single cell CB-ECFC have high proliferative potential (HPP). At least about 35% of ECFC cells or ECFC-like cells in the isolated ECFC populations provided herein are HPP-ECFCs. In certain embodiments, at least about 50% of ECFC or ECFC-like cells in the isolated ECFC populations described herein are HPP-ECFC.

In contrast, ECs produced in vitro using a protocol comprising co-culture of cells with OP9 cells (e.g., Choi et al., Stem Cells. (2009) 27(3):559-67) exhibit clonal proliferation potential wherein fewer than 3% of cells give rise to HPP-EC. Furthermore, endothelial cells produced using an in vitro protocol comprising EB formation (e.g., Cimato et al., Circulation. 2009 Apr. 28; 119(16):2170-8), have only a limited clonal proliferation potential, in which fewer than 3% of cells give rise to HPP-ECs. Endothelial cells generated in vitro from hPSCs in the presence of exogenous TGF-β inhibitors (e.g., James et al., Nat Biotechnol. (2010) 28(2):161-6), have clonal proliferation potential, where about 30% of cells give rise to HPP-ECs. However, the proliferation potential is dependent on the continued presence of TGF-β inhibition, i.e., if exogenous TGF-β inhibition is removed from this protocol the ECs lose all their HPP activity. Various techniques for measuring proliferative potential of cells are well known in the art and are described, for example, in PCT publication WO 2015/138634. Single cell assays may be used to evaluate clonogenic proliferative potential of CB-ECFCs, iPS derived-ECFCs, and EB-derived ECs. For example, proliferation potential is evaluated by culturing single cells of CB-ECFCs, ECFC-like cells or ECs in each well of a 96-well plate. Wells with an endothelial cell count of 1 are categorized as non-dividing, wells with an endothelial cell count of 2-50 are categorized as endothelial cell clusters (ECC), wells with an endothelial cell count of 51-500 or 501-2000 are categorized as low proliferative potential (LPP) cells and wells with an endothelial cell count of 2001 or greater are categorized as high proliferative potential (HPP) cells.

ECFCs have self-renewal potential. For example, the HPP-ECFCs described herein have a capacity to give rise to one or more HPP-ECFCs within a secondary HPP-ECFC colony when replated in vitro.

ECFC-like cells have self-renewal potential. For example, the HPP-ECFC-like cells described herein have a capacity to give rise to one or more HPP-ECFC-like cells within a secondary HPP-ECFC-like colony when replated in vitro.

Endothelial colony-forming cells derived using various different protocols have different capacities for blood vessel formation in vivo. For example, CB-ECFCs can form blood vessels when implanted in vivo in a mammal, such as, for example, a mouse.

In contrast, ECs produced using the protocol of Choi (Choi et al., Stem Cells. (2009) 27(3):559-67), which comprises co-culture of cells with OP9 cells for generation of EC, do not form host murine red blood cell (RBC) filled functional human blood vessels when implanted in vivo in a mammal. EC produced using the protocol of Cimato (Cimato et al., Circulation (2009) 28; 119(16):2170-8), which comprises EB formation for generation of EC, do not form host RBC filled functional human blood vessels when implanted in vivo in a mammal. EC produced using the protocol of James (James et al., Nat Biotechnol. (2010) 28(2):161-6), which comprises TGF-β inhibition for generation of EC, form significantly fewer functional human blood vessels when implanted in vivo in a mammal (i.e., 15 times fewer than cells from the presently disclosed protocol). Further the cells of James et al. can only form functional human blood vessels when implanted in vivo in a mammal if the culture continues to contain TGF-β; if TGF-β is removed the cells completely lose the ability to make RBC-filled human blood vessels. EC produced using the protocol of Samuel (Samuel et al., Proc Natl Acad Sci U S A. 2013 Jul. 30; 110(31):12774-9), which lacks the step of selecting day 12 CD31⁺NRP1⁺, can only form blood vessels when implanted in vivo in a mammal if the EC are implanted with supportive cells (i.e., mesenchymal precursor cells).

In contrast to the above prior art methods, cells in the ECFC and ECFC-like populations can form blood vessels when implanted in vivo in a mammal, even in the absence of supportive cells.

Various techniques for measuring in vivo vessel formation are known in the art (e.g., PCT publication WO 2015/138634, the content of which is incorporated herein in its entirety). For example, in vivo vessel formation may be assessed by adding the disclosed ECFCs or ECFC-like cells to three-dimensional (3D) cellularized collagen matrices A collagen mixture containing an ECFC single cell suspension is allowed to polymerize in tissue culture dishes to form gels. Cellularized gels are then implanted into the flanks of 6- to 12-week-old NOD/SCID mice. Two weeks after implantation, gels are recovered and examined for human endothelial-lined vessels perfused with mouse red blood cells. The capacity to form blood vessels in vivo in the absence of exogenous supportive cells is one indicator that the cells produced using the methods disclosed herein are ECFCs.

Cell viability may be assessed by trypan blue exclusion whereas cell senescence can be easily determined using a commercially available senescence assay kit (Biovision). ECFCs and ECFC-like cells disclosed herein have an enhanced cell viability and/or reduced senescence relative to CB-ECFCs or ECs produced by alternative means. For example, ECs produced using the protocol of Choi et al (2009), which comprises co-culture of cells with OP9 cells, have a lower cell viability of only 6 passages. ECs produced using the protocol of Cimato (Cimato et al., Circulation (2009) 28; 119(16):2170-8), which requires EB formation, have a lower cell viability of only 7 passages. ECs produced using the protocol of James (James et al., Nat Biotechnol. (2010) 28(2):161-6), which requires exogenous TGF-β inhibition, have a cell viability of 9 passages. Moreover, removal of the TGF-β inhibition, leads to a loss of the endothelial cell phenotype and a transition to a mesenchymal cell type. ECs produced using the protocol of Samuel (Samuel et al., Proc Natl Acad Sci U S A. 2013 Jul. 30; 110(31):12774-9) which lacks the step of selecting day 12 CD31⁺NRP-1⁺ cells, can be expanded for up to 15 passages. In contrast to the above methods for generating ECs in vitro, ECFCs produced by the methods disclosed herein can be expanded for up to 18 passages whereas CB-ECFCs can be passaged from between 15 and 18 times.

### Therapeutic Uses of Compositions Comprising ECFCs and ECFC-like Compositions

In certain embodiments, the pharmaceutical compositions provided herein comprise serum-free chemically defined media conditioned by ECFCs or and ECFC-like cells useful for treating perfusion disorders in tissues, organs or extremities of a subject in need thereof

As described herein, ECFCs can be obtained from various sources, such as, for example, pluripotent stem cells expressing at least one stem cell transcription factor, e.g. OCT-4A, NANOG or SOX2, including, but not limited to, embryonic stem cells (ESCs), primordial germ cells (PGCs), adult stem cells, or induced pluripotent stem cells (iPSCs). In certain embodiments, the ECFCs can be obtained from umbilical cord blood stem cells. In other embodiments, ECFC-like cells can be generated through the endothelial cell differentiation of KDR⁺NCAM⁺APLNR⁺ mesodermal (MSD) precursor cells.

ECFCs or ECFC-like cells can be cultured in a cell culture medium, in vitro. After a period of time in culture, ECFCs or ECFC-like cells can be washed and incubated in a chemically defined medium (CDM). In certain embodiments, the ECFCs or ECFC-like cells are cultured to near confluency prior to be being y irradiated or treated with mitomycin C to arrest cell division. The cells are then thoroughly washed and fresh CDM is added. After about 24-48 hours, the medium is harvested, and any residual cells are removed by filtration or centrifugation. This medium, conditioned by the cultured ECFCs or ECFC-like cells, is referred to as ECFC-conditioned medium (ECFC-CM) or ECFC-like CM respectively, and contains various components secreted by the ECFCs or ECFC-like cells, including microvesicles, extracellular vesicles (EV) and/or the ECFC or ECFC-like cells exosomes. In certain embodiments, the chemically defined medium can be conditioned with ECFCs or ECFC-like cells for 20 minutes to 48 hours, 20 minutes to 36 hours, 20 minutes to 24 hours, 20 minutes to 12 hours or 20 minutes to 6 hours. In certain embodiments, the medium is conditioned with the ECFCs or ECFC-like cells for approximately 2-5 days. In certain embodiments, the γ irradiated or mitomycin treated cells are cultured as a monolayer in semi-permeable Corning® Transwell® inserts.

Compositions suitable for use with the methods disclosed herein may comprise all or a portion of ECFC-CM or ECFC-like CM. For example, ECFC-CM or ECFC-like CM may be perfused into a tissue, without further modification. Alternatively, the ECFC-CM or ECFC-like CM may be diluted, concentrated (e.g. using an EMD Millipore Amicon Centrifugal Filter), or separated to obtain a specific fraction, or combined with one or more other compounds or compositions, such as, for example a solution for transporting and/or preserving an organ (e.g., UW solution, Stanford solution, Steen solution etc.). In certain embodiments, the compositions provided herein may be supplemented with one or more angiogenic factors. In certain embodiments, the compositions provided herein comprise extracellular vesicles (EVs) separated from ECFC-CM or ECFC-like CM. EVs contain cargos of factors that may be unstable in the extracellular milieu, such as microRNAs.

Exemplary methods of making conditioned media and administration of same or fraction thereof can be found, for example, in the published U.S. Patent Application 2006/0165667, the content of which is incorporated herein by reference in its entirety.

In certain embodiments, the ECFCs or ECFC-like cells used to condition the chemically defined medium (CDM) may be “preconditioned” by one or more treatments.

In certain embodiments, a pretreatment step may comprise or consist of culturing the ECFCs or ECFC-like cells on an extracellular matrix protein and/or peptide. In certain embodiments, the extracellular matrix proteins and/or peptides serve to precondition the ECFCs or ECFC-like cells for anticipation of in vivo microenvironment or microenvironments. In certain embodiments, the extracellular matrix proteins and/or peptides may be comprised of molecules that are capable of modulating the biophysical properties to change the elasticity of the substrate extracellular matrix proteins and/or peptides. In certain embodiments, the extracellular matrix protein is type 1 collagen, fibronectin, vitronectin, or peptides that are generated specifically to interact with cell surface receptors on the ECFCs or ECFC-like cells. In certain embodiments, the pretreatment step may comprise or consist of lowering the tissue culture oxygen concentration to 1% and placing the ECFC or ECFC-like cells under arterial or venous simulated laminar flow conditions.

Preserving and/or improving endothelial function in organs and tissues is important for mitigating and/or preventing ischemic injury and/or reperfusion injury. Preserving and/or improving endothelial function in organs and tissues can reduce vascular injury and/or promote vascular repair in the injured tissues and organs.

In certain embodiments, the disclosure provides for endothelial colony-forming cells (ECFCs) and/or a secretion from endothelial colony-forming cells (ECFCs) and/or at least a fraction of endothelial colony-forming cells-conditioned medium (ECFC-CM) (referred to herein as an “ECFC composition”), can be used for the treatment or prophylaxis of a perfusion disorder in a subject, or to preserve (at least in part) and/or rescue (at least in part) tissue from ischemic and/or reperfusion injury. ECFCs may mitigate inflammation in ischemic tissue, reduce the release of reactive oxygen species, prevent apoptosis and/or promote angiogenesis, and/or the proliferation of endogenous stem-like cells.

In certain embodiments, the disclosure further provides for endothelial colony-forming like cells (ECFC-like cells) and/or a secretion from endothelial colony-forming like cells (ECFC like cells) and/or at least a fraction of endothelial colony-forming like cells-conditioned medium (ECFC-like CM) (referred to herein as “ECFC-like compositions”), can be used for the treatment or prophylaxis of a perfusion disorder in a subject, or to preserve (at least in part) and/or rescue (at least in part) tissue from ischemic and/or reperfusion injury. ECFCs may mitigate inflammation in ischemic tissue, reduce the release of reactive oxygen species, prevent apoptosis and/or promote angiogenesis and/or the proliferation of endogenous stem-like cells.

The materials and methods provided herein are applicable to a variety of tissues, organs or extremities (e.g., of a subject), in a variety of functional states (e.g., abnormal tissue/organ function, such as impaired function). For example, tissues and organs characterized by being susceptible to ischemia and hypoxia-induced progressive cell damage are suitable for use with the compositions and methods provided herein. For example, the materials and methods provided herein can be used to treat ischemia in mesenteric tissue, cardiac tissue, lung tissue, cerebral tissue, liver tissue, and/ or renal tissue; or organs such as the heart, lung, brain, liver or kidney.

In one embodiment, the compositions and methods treat a tissue or organ by preserving and/or improving endothelial function in the tissue or organ. In other embodiments, the compositions and methods treat a tissue or organ by reducing vascular injury or by promoting vascular repair in the tissue or organ. Methods for assessing endothelial function, and vascular injury and repair are known in the art and are provided herein.

As described further below, administration of an ECFC or ECFC-like composition into adult, infant, or neonatal kidneys protects the kidneys (at least in part) from loss of function caused by ischemic injury and/or reperfusion injury. At least some of the compounds secrete into the cell culture medium by ECFCs or ECFC-like cells provide a protective and/or restorative effect on adult, infant, and neonatal kidney tissue.

As discussed below, various concentrations of an ECFC composition or ECFC-like composition, such as ECFC-CM or ECFC-like CM, can be used to treat human or animal subjects before, during or after the subject undergoes an ischemic event. The event may be, for example, a mesenteric ischemia-reperfusion event, a myocardial ischemia-reperfusion event, a lung ischemia-reperfusion event, a cerebral ischemia-reperfusion event, a liver ischemia-reperfusion event or a kidney ischemia-reperfusion event. It is also contemplated that the ECFC and ECFC-like compositions provided herein can be used to reduce or prevent reperfusion damage to adult, infant, or neonatal tissue. Pre-treatment of the ECFCs or ECFC-like cells used to condition the ECFC-CM or ECFC-like CM respectively may also improve treatment of the tissue before, during, and/or after an ischemic and/or reperfusion event.

In certain embodiments, a method of treating a tissue with an ECFC or ECFC-like composition is provided. For example, a tissue may be perfused with an ECFC or ECFC-like composition disclosed herein, for a period of time, thereby preventing or mitigating a perfusion disorder, such as ischemic and/or reperfusion injury of the tissue or rescuing the tissue from ischemic and/or reperfusion injury. Various systems for perfusing tissues and organs are known, such as, for example, the Langendorff system or a tissue/organ bath system.

In an embodiment, the compositions provided herein may be delivered to a site in a subject other than the tissue or organ to be treated. For example, an ECFC or ECFC-like composition can be administered to the subject experiencing tissue or organ damage, for example, as a result of ischemic and/or reperfusion injury. The ECFC or ECFC-like composition can be administered at a site other than the injured tissue or organ, for example, at a site adjacent to or near the injured tissue or organ. Soluble factors produced by the ECFC or ECFC-like cells can be released from the ECFCs or ECFC-like cells and act on the injured tissue or organ.

In an embodiment, the tissue or organ may be treated ex vivo. For example, in various organ or tissue transplant systems, the donor organ/tissue is maintained ex vivo for a period of time. During this time, there is inadequate blood flow to the organ, and consequently inadequate oxygen supply to the organ. This period of ischemia (also referred to herein as an ischemic event) damages the organ. When blood supply returns to the tissue (i.e., reperfusion), after the ischemic event, it can injure the tissue, for example by causing inflammation and oxidative stress, rather than restoring normal tissue function.

In certain embodiments, the tissue or organ may be treated in situ. For example, following acute kidney injury, which damages renal tissue, the compositions provided herein may be delivered to the injured kidney to preserve and/or improve endothelial function in the kidney and/or to reduce vascular injury in the kidney and/or to promote vascular repair in the kidney.

In various aspects of the method provided, perfusion of tissue with a composition comprising ECFCs, ECFC-CM or fraction thereof, ECFC-like cells or ECFC-like CM or fraction thereof may be carried out before, during and/or after an ischemic event. In an embodiment, treatment may be systemic, wherein the ECFC or ECFC-like composition is provided to the patient systemically, or locally. Alternatively, or additionally, in an embodiment, the perfusion may be carried out before and/or during reperfusion.

Perfusion with a composition comprising ECFCs or ECFC-like cells, as provided herein, may be carried out at various doses over various time periods. For example, a composition comprising ECFCs or ECFC-like cells may contain about 10⁴, 10⁵, 10⁶, 10⁷ or 10⁸ ECFCs or ECFC-like cells/ml and may be provided to a tissue or organ in need thereof e.g. by perfusion before, during and/or after ischemia.

In certain embodiments, a minimum of about 10⁴ ECFC or ECFC-like cells/ml are provided or administered to a tissue or organ. In certain embodiments, a minimum of about 10⁵ ECFC or ECFC-like cells/ml are provided or administered to a tissue or organ. In certain embodiments, a minimum of about 10⁶ ECFC or ECFC-like cells/ml are provided or administered to a tissue or organ. In certain embodiments, a minimum of about 10⁷ ECFC or ECFC-like cells/ml are provided or administered to a tissue or organ. In certain embodiments, a minimum of about 10⁸ ECFC or ECFC-like cells/ml are provided or administered to a tissue or organ. In certain embodiments, a range of between 10⁴ and 10⁶ ECFC or ECFC-like cells/ml are provided or administered to a tissue or organ. In certain embodiments, a range of between 10⁵ and 10⁷ ECFC or ECFC-like cells/ml are provided or administered to a tissue or organ. In certain embodiments, a range of between 10⁶ and 10⁸ ECFC or ECFC-like cells/ml are provided or administered to a tissue or organ. In certain embodiments, a range of between 10⁶ and 10⁷ ECFC or ECFC-like cells/ml are provided or administered to a tissue or organ. In certain embodiments, a range of between 10⁷ and 10⁸ ECFC or ECFC-like cells/ml are provided or administered to a tissue or organ.

Perfusion with a suitable ECFC or ECFC-like composition, as provided herein, may be carried out at various doses over various time periods. For example, ECFC-CM or ECFC-like CM or fractions thereof may be provided to a tissue or organ to be treated at a therapeutically effective concentration (e.g., at a concentration of about 1, 5, 10, 50, 100 or 200 ng/ml total protein) before, during and/or after ischemia. In various embodiments, the ECFC or ECFC-like composition is provided as an adjunct to treatment with an organ transport/preservation solution, such as UW solution, Stanford solution, Steen solution, etc.

Results of tissue treatment with an ECFC and/or ECFC-like composition, as provided herein, may be measured in a variety of ways, such as, for example, by functional assay (i.e., to determine one or more indicator of tissue/organ function), or molecular assay (i.e., to determine one or more molecular feature of the tissue/organ).

In one embodiment, one or more functional assay is used to determine results of the treatment, wherein results of the functional assay are compared to a standard. For example, the standard for a functional assay may be indicative or a normally functioning tissue/organ, or an abnormally functioning tissue/organ (e.g., a tissue/organ having impaired function).

### Conditions to be Treated Using ECFC or ECFC-like Compositions Ischemic-Reperfusion Event.

An ECFC or ECFC-like composition can be used to treat a number of conditions, diseases and disorders. In an embodiment, the compositions can be used to treat an ischemic-reperfusion (FR) event. Although restoration of blood flow to an ischemic tissue or organ is essential to preventing further tissue/organ damage, reperfusion itself can also damage the tissue/organ. For example, I/R events affect the vasculature of the tissue, and in particular damages the vascular endothelium. This results in impaired vascular function, for example, by reducing blood flow though the tissue or organ, altering vascular tone and/or increasing inflammatory responses. I/R events can occur in a variety of situations, including, for example, including reperfusion after thrombolytic therapy, coronary angioplasty, organ transplantation, or cardiopulmonary bypass. Consequently, a number of different tissues and organs may be affected by I/R events, including, for example, mesenteric tissue, cardiac tissue, lung tissue, cerebral tissue, liver tissue, kidney tissue; as well as hearts, lungs, brains, livers and kidneys.

In certain embodiments the ECFC or ECFC-like compositions disclosed herein can be used to treat peripheral artery disease and critical limb ischemia (CLI).

An ECFC or ECFC-like composition may be used to preserve and/or improve endothelial function. In certain embodiments, endothelial function is preserved relative to a tissue or organ that does not receive the ECFC or ECFC-like composition. In an embodiment, endothelial function is improved by about 5%,10%, 15%, 20%, 25%, 30%, 35%, 40%, 45%, 50%, 55%, 60%, 65%, 70%, 75%, 80%, 85, 90%, or 95% relative to a tissue or organ that does not receive the ECFC or ECFC-like composition. In certain embodiments, the endothelial function is improved be greater than 10%, greater than 20%, greater than 30%, greater than 40%, greater than 50%, greater than 60%, greater than 70%, greater than 80%, greater than 90%, greater than 95% or greater than 99% relative to a tissue or organ that did not receive the ECFC or ECFC-like composition.

An ECFC or ECFC-like composition may be used to reduce vascular injury to the tissue or organ in association with an FR event. In an embodiment, the vascular injury is reduced relative to a tissue or organ that does not receive the composition comprising ECFC or an ECFC composition. In an embodiment, the vascular injury is reduced by about 5%, 10%, 15%, 20%, 25%, 30%, 35%, 40%, 45%, 50%, 55%, 60%, 65%, 70%, 75%, 80%, 85, 90%, or 95% relative to a tissue or organ that does not receive the composition comprising ECFC or an ECFC composition. In an embodiment, the vascular injury is reduced by greater than 10%, greater than 20%, greater than 30%, greater than 40%, greater than 50%, greater than 60%, greater than 70%, greater than 80%, greater than 90%, greater than 95% or greater than 99% relative to a tissue or organ that did not receive the ECFC or ECFC-like composition.

An ECFC or ECFC-like composition may be used to promote or increase vascular repair in the tissue or organ in connection with an I/R event. In an embodiment, the vascular repair is increased relative to a tissue or organ that does not receive the composition comprising ECFC or an ECFC composition. In an embodiment, the vascular repair is increased by about 5%,10%, 15%, 20%, 25%, 30%, 35%, 40%, 45%, 50%, 55%, 60%, 65%, 70%, 75%, 80%, 85, 90%, or 95% relative to a tissue or organ that does not receive the composition comprising ECFC or an ECFC composition. In an embodiment, the vascular repair is increased by greater than 10%, greater than 20%, greater than 30%, greater than 40%, greater than 50%, greater than 60%, greater than 70%, greater than 80%, greater than 90%, greater than 95% or greater than 99% relative to a tissue or organ that did not receive the ECFC or ECFC-like composition.

An ECFC or ECFC-like composition may be used to preserve medullary blood flow in a post-ischemic tissue or organ. In certain embodiments, medullary blood flow is preserved relative to a tissue or organ that does not receive the composition comprising ECFCs or an ECFC composition.

An ECFC or ECFC-like composition may be used to reduce infiltration of inflammatory cells in an organ or tissue injured in association with an I/R event. In an embodiment, the infiltration of inflammatory cells is reduced relative to a tissue or organ that does not receive the composition comprising ECFC or an ECFC composition. In an embodiment, the infiltration of inflammatory cells is reduced by 5%,10%, 15%, 20%, 25%, 30%, 35%, 40%, 45%, 50%, 55%, 60%, 65%, 70%, 75%, 80%, 85, 90%, or 95% relative to a tissue or organ that does not receive the composition comprising ECFC or an ECFC composition. In an embodiment, the infiltration of inflammatory cells is reduced by greater than 10%, greater than 20%, greater than 30%, greater than 40%, greater than 50%, greater than 60%, greater than 70%, greater than 80%, greater than 90%, greater than 95% or greater than 99% relative to a tissue or organ that did not receive the ECFC or ECFC-like composition.

In an embodiment, the organ or tissue to be treated is a transplanted organ or tissue that is ischemic and then reperfused or a tissue or organ that is being prepared for transplantation. Contact between the tissue or organ and an ECFC or ECFC-like composition protects (at least in part) and/or reverses (at least in part) ischemic and/or reperfusion injury of the tissue or organ, thereby preparing the tissue such that it is suitable or more suitable for transplantation.

In an embodiment, the organ or tissue to be treated is an organ or tissue that is damaged due to exposure to ionizing radiation. Tissues that have been irradiated experience I/R injuries induced by, for example, reactive oxygen species. An ECFC or ECFC-like composition protects (at least in part) and/or reverses (at least in part) ischemic and/or reperfusion injury of the irradiated tissue or organ, thereby helping the tissue to recover and/or to recover faster.

In certain embodiments, the ECFC or ECFC-like composition is used to treat a renal ischemic-reperfusion (I/R) event. In a renal I/R event, vascular function is impaired due to reduced renal blood flow and glomerular filtration while promoting parenchymal cell damage and sustained injury. Renal endothelium is an important target in the injury process. This endothelium damage may compromise renal blood flow by imparting changes in vascular tone and/or increasing inflammatory responses. In addition to acute endothelial dysfunction, there is a significant reduction in peritubular capillary density following acute kidney injury (AKI). This reduction in peritubular capillary density is characterized by low endothelial cell proliferation and propensity to undergo endothelial-to-mesenchymal transition. The ECFC or ECFC-like composition can be used to preserve and/or improved endothelial function protect the vasculature in the kidney or to promote revascularization. The ECFC or ECFC-like composition may also be used to reduce vascular injury and/or to promote vascular repair. The ECFC or ECFC-like composition may also be used to decrease loss in renal medullary perfusion; protect against impaired renal blood flow and/or preserve hemodynamic function post-ischemia. The treatment can be in a subject in need of such treatment, for example a subject with acute kidney injury or in a subject having undergone, undergoing or about to undergo a renal ischemia-reperfusion event.

The ECFC or ECFC-like composition may also be used to reduce post-ischemic endothelial leukocyte adhesion in a subject in need thereof, for example, a subject with acute kidney injury or in a subject having undergone, undergoing or about to undergo a renal ischemia-reperfusion event. In certain embodiments, the post-ischemic endothelial leukocyte adhesion is mediated by ICAM-1, an adhesion molecule known to be induced in endothelial cells in the post-ischemic period. In certain embodiments, the leukocyte adhesion is mediated by VCAM-1. In certain embodiments, the leukocyte adhesion is mediated by PECAM-1. In certain embodiments, the leukocyte adhesion is mediated by a selectin such as E-Selectin or P-Selectin. In certain embodiments, the leukocyte adhesion is mediated by a β2-integrin such as LFA-1 (CD11a/CD18) or Mac-1 (CD11b/CD18). In one embodiment, the leukocyte adhesion is mediated by two or more molecules chosen from ICAM1, VCAM-1 PECAM-1 E-Selectin, P-Selectin, LFA-1 and Mac-1. In one embodiment, the leukocyte adhesion is mediated by three or more molecules chosen from ICAM1, VCAM-1 PECAM-1 E-Selectin, P-Selectin, LFA-1 and Mac-1.

The ECFC or ECFC-like composition may also be used to reduce post-ischemic inflammation in a subject in need thereof, for example, a subject with acute kidney injury or in a subject having undergone, undergoing or about to undergo a renal ischemia-reperfusion event. In an embodiment the specific anti-inflammatory cells population are reduced upon administration of the ECFC or ECFC-like composition. In certain embodiments, the cell population is a population expressing the cytokine IL-17, T-helper 17 cells (i.e., CD4+/IL-17+) or Th-1 cells (i.e., CD4+/IFN-γ+). The ECFC or ECFC-like composition may also be used to reduce infiltration of one or more of these cell populations in a subject in need thereof, for example, a subject with acute kidney injury or in a subject having undergone, undergoing or about to undergo a renal ischemia-reperfusion event.

### Kits

The present disclosure contemplates kits for carrying out the methods disclosed herein. Such kits comprise two or more components required for treatment of a tissue or organ, as provided herein. Components of the kit include, but are not limited to, an ECFC or ECFC-like composition, and one or more of compounds, reagents, containers, equipment, and instructions for using the kit. Accordingly, the methods described herein may be performed by utilizing pre-packaged kits provided herein. In one embodiment, the kit comprises an ECFC or ECFC-like composition and instructions. In some embodiments, the instructions comprise one or more protocols for preparing and/or using the ECFC or ECFC-like composition in the method provided herein. In some embodiments, the kit comprises one or more reagents for performing a functional assay (to determine one or more indicators of tissue/organ function), or a molecular assay (to determine one or more molecular features of the tissue/organ) and instructions comprising one or more protocols for performing such assays, such as, for example, instructions for comparison to one or more standards. In some embodiments, the kit comprises one or more standards (e.g., standard comprising a biological sample, or representative transcript expression data).

In one embodiment, the kit comprises ECFC-CM or ECFC-like CM, as described herein. By way of example, the kit may contain a container comprising one or more doses of ECFC-CM or ECFC-like CM and instructions for their use. In a preferred embodiment, the kit may further comprise one or more organ transplant/preservation compositions, such as UW solution, Stanford solution, Steen solution etc.

### EXAMPLES

### Exemplary Embodiments

The disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Methods and Materials

### Animals

Male Sprague-Dawley rats (initial weight ˜250 g) were utilized in all studies. Rats were given free access to standard rat chow and water throughout our studies. Experiments were conducted in accordance with National Institutes of Health guidelines and were approved by the Indiana University School of Medicine Institutional Animal Care and Use Committee.

### Cells

Rat pulmonary microvascular endothelial cells (PMVEC) and rat pulmonary artery cells (PAEC) were isolated and expanded as described previously (Alvarez et al., Am J Physiol Lung Cell Mol Physiol 294: L419 —L430, 2007). These primary cultures were derived from Sprague Dawley rats and utilized between passages 5 and 7. The endothelial nature of PMVEC and PAEC was previously characterized by Alvarez (Alvarez et al., 2007) and cells were validated according to their expression of CD31, KDR, and vWF, but were negative for CD45 and CD133. PMVEC have a significantly faster proliferation rate and a greater percentage of high proliferative potential HPP-ECFC than PAEC (Alvarez et al., 2007). PMVEC and PAEC were maintained in EGM-2 supplemented with 10% FBS (Hyclone) and grown on T75 flasks. On the day of transplant studies, cells were harvested by trypsin digestion, washed with PBS. In some studies, the cells were labeled with CMTPX (i.e., Cell tracker red, Invitrogen), according to the manufacturer's instructions. The cells were then washed and resuspended in serum-free culture medium and maintained on ice until the time of transplant.

Human ECFCs were derived from human cord blood according to the protocol described previously by Yoder et al. (Yoder et al., Blood 109: 1801-1809, 2007). Human ECFCs were maintained in T-225 flasks in EGM2 (Invitrogen) with 10% FBS. Fifty milliliters of conditioned serum-free medium was derived from 50 to 75% confluent human ECFCs, corresponding to ˜8-12 million cells following 2 days of incubation and concentrated by centrifugation using Centricon filters (3000 M.W. cutoff) to achieve an enrichment of ˜10-fold. Therefore, 1 ml of conditioned medium (ECFC-CM) results from the contribution of ˜1.6-2.4 million cells.

### Surgeries

Acute kidney injury was induced by bilateral ischemia reperfusion injury to the kidneys by clamping both renal pedicles for 40 min using a surgical approach that has been described previously under anesthesia induced with ketamine (100 mg/kg) and pentobarbital (25-50 mg/kg) (Phillips et al., Am J Physiol Regul Integr Comp Physiol 298: R1682—R1691, 2010) or ketamine (100 mg/kg) and xylazine (5 mg/kg). The first cocktail was used in the initial series of experiments in which rat ECFCs were tested; while the second anesthetic cocktail was used in studies of human ECFC derived conditioned media. The reason for the change was due to limited availability of pentobarbital which occurred between the times of the two studies. These two anesthetic regimens yielded consistent levels of renal injury.

For endothelial cell administration, an approach similar to that described by Brodsky et al., for the administration of HUVEC (Brodsky et al., Am J Physiol Renal Physiol 282: F1140-F1149, 2002) was utilized. The left carotid artery was cannulated with a PE-50 tubing filled with heparinized sterile saline, inserted toward the heart, while the artery distal to the insertion site was ligated with a silk-suture to prevent backleak. This catheter was utilized for the administration of cells (5×10⁶ PMVEC or PAEC in 0.5 ml of vehicle) in a retrograde fashion immediately following the release of the clamps. The catheter was then slowly withdrawn, and the carotid artery was immediately ligated proximal to the insertion site to prevent bleeding. In studies using ECFC-conditioned media, a volume of 0.5 ml of 10× concentrated conditioned media or “mock”-conditioned media from human ECFCs was administered to the suprarenal aorta at the time of reperfusion using a 31-gauge needle.

### Measurement of Renal Function

At the indicated times, blood was obtained from rats under light isoflurane anesthesia via tail vein incisions. Blood was collected in 1.5-ml heparinized Eppendorf tubes and centrifuged at 3,000 g for 10 min. Serum creatinine was measured using a Point Scientific QT 180 Analyzer and creatinine reagent kit (Point Scientific, Canton, Mich.) according to the manufacturer's specifications (Vella F. Textbook of Clinical Chemistry. Tietz N W, Editor. Philadelphia, Pa.: Saunders, 1986).

### Evaluation of KIM-1 or ICAM-1 mRNA Expression in the Injured Kidney

Whole kidney mRNA was extracted from fresh-frozen tissue using a Direct-zol RNA extraction kit according to the manufacturer's instructions (Zymo, Irvine, Calif.). Kidney injury molecule-1 (KIM-1) mRNA expression was evaluated using predesigned Taqman primers (Life Technologies, Carlsbad, Calif.) with the 2^{-ΔΔ}C_{T} analysis method (Livak et al., Method. Methods 25: 402-408, 2001).

### Evaluation of Renal Hemodynamic Response to I/R Injury

Rats were anesthetized with ketamine HCl (60 mg/kg), followed by Inactin (50-100 mg/kg) intraperitoneal injection and placed on a heated surgical board to maintain body temperature at 37° C. The femoral vein was cannulated for intravenous infusion of 2% bovine serum albumin in 0.9% NaCl at a rate of 2 ml·h⁻¹·100 g body wt⁻¹. This catheter was also used for infusion of conditioned medium.

A midline abdominal incision was made, and a flow probe was placed around the renal artery for measurement of renal blood flow (RBF) via an ultrasonic Doppler flowmeter (model T206; Transonic Systems, Ithaca, N.Y.). The left kidney was placed in a holder and an optical probe for laser Doppler flowmetry (Transonic) was implanted to a depth of ˜5.0 mm beneath the surface for measurements of renal outer medullary blood flow (MBF). Data were recorded using Biopac (Goleta, Calif.) data-acquisition software.

Following 30 min of equilibration, RBF and MBF values were measured for 30 min in 10-min time bins, with the final 10 min defined as baseline. Parameters were measured during ischemia and an additional 120 min of reperfusion. Values were normalized to each baseline value, and data are expressed as the average of these normalized values.

### Evaluation of Cell Homing

Prior to transplant, rat PMVEC were stained with cell tracker red CMTPX, as described above. Pilot studies indicated that tissue fixation impaired the detection of labeled cells. Therefore, cell fluorescence was examined in freshly harvested unfixed tissues. Kidneys, spleens, or lungs were removed from deeply anesthetized rats and immersed in ice-cold HEPES-Tyrode buffer (132 mM NaCl, 4 mM KCl, 1 mM CaCl₂, 0.5 mM MgCl₂, 10 mM HEPES and 5 mM glucose, pH 7.4) that had been bubbled with 100% O₂. Tissue slices were prepared using a hand microtome (Stadie Riggs Tissue Slicer), stored in cold buffer and imaged within 1 h of tissue harvest. Images were obtained using a Zeiss LSM NLO confocal microscope equipped with Ar and HeNe lasers and a X40 water immersion lens, and a signal was obtained by 545 nm and detection at 565-615 nm.

### Evaluation of Infiltrating Leukocytes

Harvested kidneys were minced and digested in TL Liberase (2 μg/ml; Roche). The obtained cell suspension was filtered through a 100-μm filter mesh and washed with DMEM containing 10% fetal bovine serum (Cell Applications, San Diego, Calif.). The mononuclear cells were separated by Percoll (Sigma, St. Louis, Mo.) and counted by hemocytometer. To evaluate T lymphocytes, the cells were stained with antibodies against rat CD4 (PE-Cy7: BD Biolgend, San Diego, Calif.), CD8a (Alexa 647: BD Biolgend). To evaluate the cytokines secreted by T cells, the cells were stained for the CD4 surface marker, permeabilized using 0.1% saponin and stained with antibodies against rat IFN-γ (FITC: BD Biolgend) or IL-17 (FITC: BD Biolgend). Cells were scanned using flow cytometry (FACSCalibur, BD Biosciences), and scans were analyzed using Flowjo software (Tree Star, Ashland, Oreg.). The gating strategy used for these analyses was exactly as previously described (Mehrotra et al., Kidney Int 88: 776-784, 2015). The total numbers of the different T cell populations in the harvested kidney were calculated using the percentage of each cell type and the total cell number measured per gram of kidney.

### Renal Histology and Immunohistochemistry

Renal tubular damage was evaluated from formalin-fixed, paraffin-embedded samples stained using periodic acid-Schiff (PAS). Six random images (3 cortex, 3 outer medulla) were obtained using a Leica DMLB microscope (Scientific Instruments, Columbus, Ohio) using a X20 objective. For each kidney, an average of 60 tubules were scored from images by an observer who was blinded to the treatments using a 1-4 scoring system described previously (Basile et al., Kidney Int 83: 242-250, 2013). Data presented are based on the average score per tubule corresponding to each animal.

### Immunofluorescent Analysis of ICAM-1

Methanol-fixed 100-μm vibratome sections of kidneys were subjected to immunofluorescent staining using an anti-ICAM-1 antibody (BD Biosciences, San Jose, Calif.). ICAM-1-specific signals were developed using a tyramide signal amplification kit (Invitrogen, Carlsbad, Calif.) as described previously (Basile et al., Am J Physiol Renal Physiol 300: F721-F733, 2011). Confocal images were obtained using an Olympus FV 1000-MPE microscope using a X20 objective (Center Valley, Pa.). Quantification of immunofluorescence was done with the aid of Fiji ImageJ. Data presented are based on the % total ICAM-1-stained area.

### Statistical Analysis

Data are expressed as means±SE. Differences in means were established by Student's t-test or ANOVA as indicated. The 0.05 level of probability was utilized as the minimum criterion of significance. All statistical analyses were performed using GraphPad Prism 6.0 (GraphPad Software, La Jolla, Calif.).

### Example 2: Rat PMVEC Protect Against Renal Ischemia-Reperfusion (I/R) Injury and Accelerate Functional and Structural Recovery

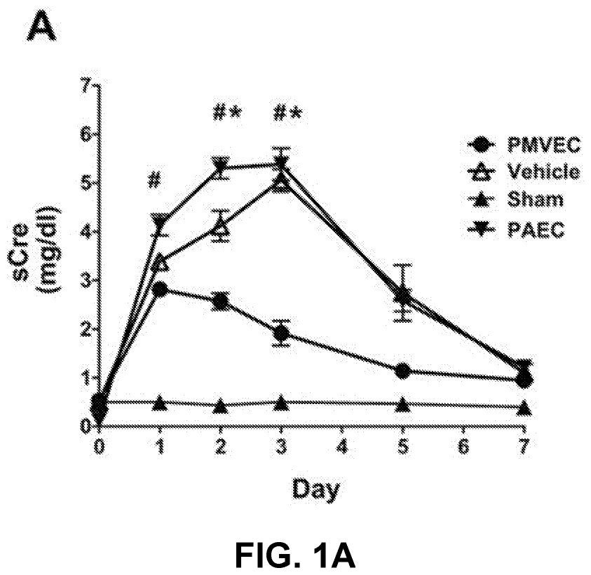

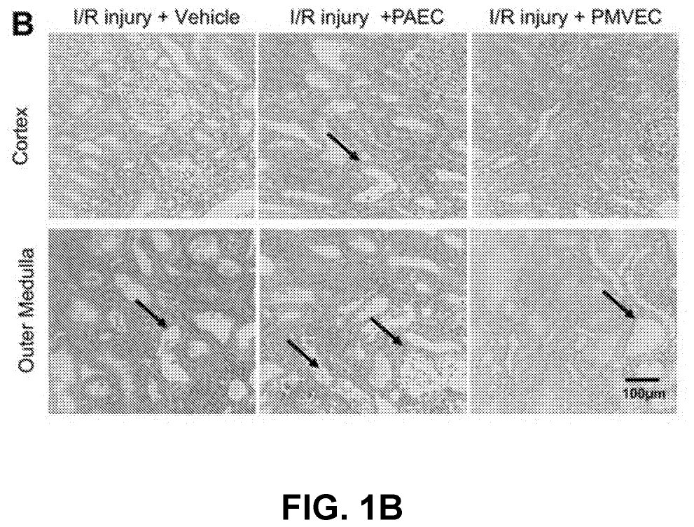

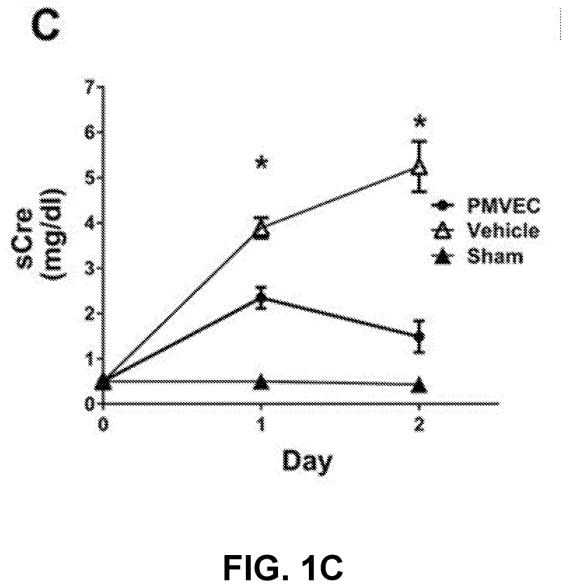

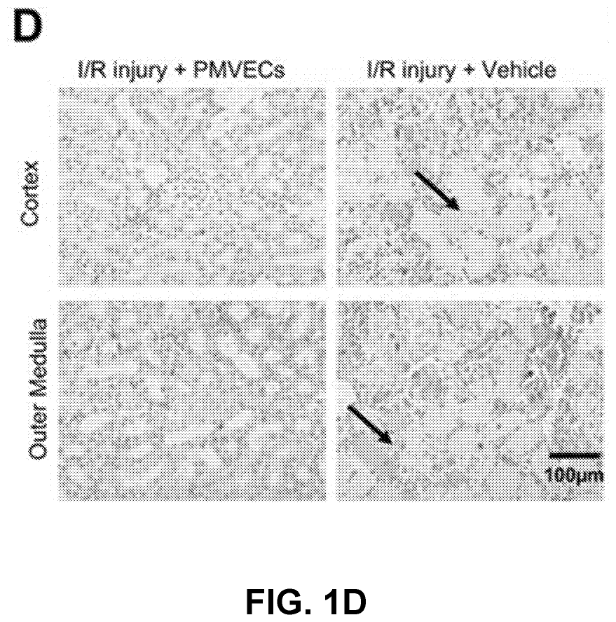

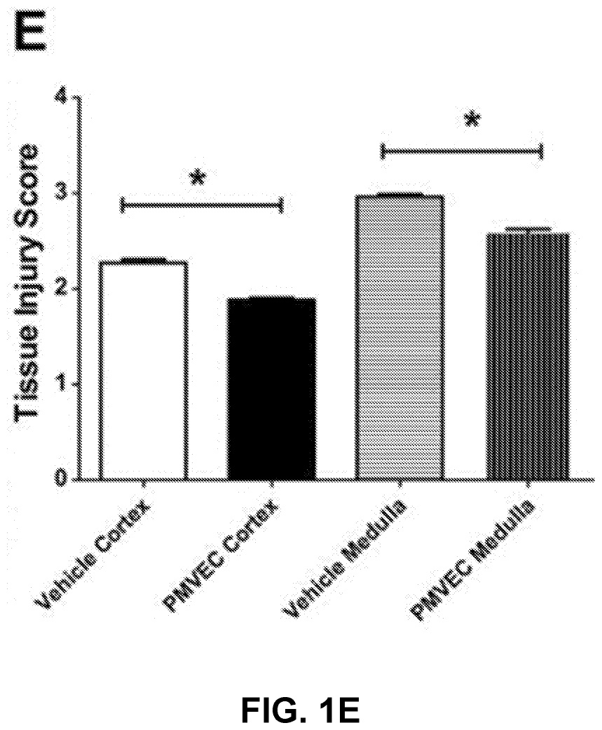

The potential that ECFCs may alter the course of renal dysfunction and/or repopulate the renal microvasculature as a function of proliferative potential was addressed by comparing the effect of administered rat PMVEC, which have a high percentage of HPP-ECFCs, or rat PAECs, which have a low percentage of HPP-ECFCs (Alvarez et al., Am J Physiol Lung Cell Mol Physiol 294: L419-L430, 2007). Renal injury measured by increased serum creatinine was most prominent at 2 days of reperfusion. Relative to vehicle-treated control rats, PMVEC-treated rats had a lower peak creatinine level and a faster recovery of serum creatinine levels (FIG. 1A). In contrast, PAEC administration did not alter the course of renal injury relative to vehicle-treated rats. Despite evidence of recovery in all groups, the level of histological damage remained severe in post-ischemic, vehicle-treated animals at day 7 with evidence of sloughed cells and tubular dilatation in the outer medulla (black arrows, FIG. 1B) compared with PMVEC-treated rats (FIG. 1B). To further investigate the protective effect of PMVEC, additional animals were studied at 2 days following reperfusion. Similar to FIG. 1A, PMVEC-treated rats had lower peak serum creatinine levels (FIG. 1C) and reduced necrotic damage compared with vehicle-treated, post-ischemic rats (black arrows; FIG. 1D and FIG. 1E).

### Example 3: Rat PMVEC Preserve Medullary Blood Flow in the Early Post-Ischemic Period

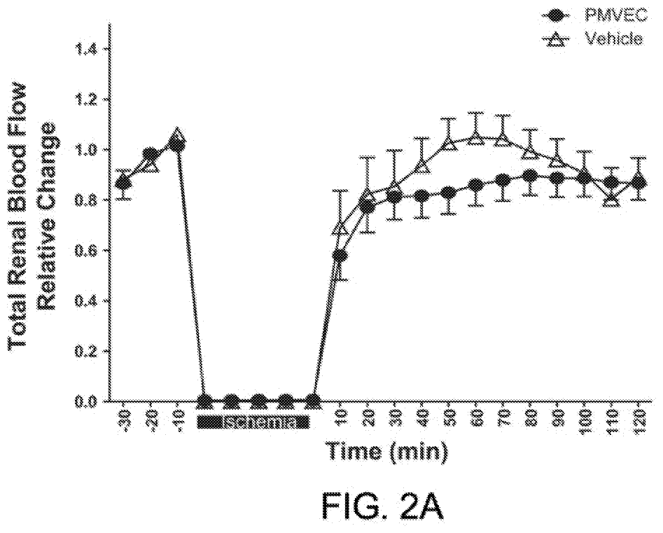

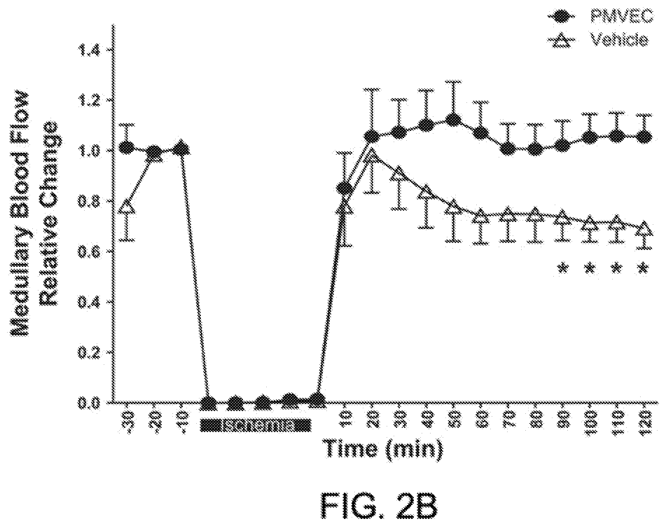

To investigate the potential mechanism of PMVEC-mediated protection, the influence of these cells on hemodynamic function in the early post-ischemic period was investigated by measuring total RBF and outer MBF following reperfusion. Total RBF values rapidly recovered during the reperfusion phase and were similar to baseline values within 30-40 min. At 2 h of reperfusion, total RBF was ˜90-95% of baseline in both vehicle-treated and PMVEC-treated animals (not significant; FIG. 2A). In contrast, MBF gradually declined over the course of 2 h following reperfusion in vehicle-treated rats. However, PMVEC-treated rats had significantly preserved MBF relative to vehicle-treated rats (FIG. 2B).

### Example 4: Rat PMVEC Do Not Home to the Kidney Following Transplantation

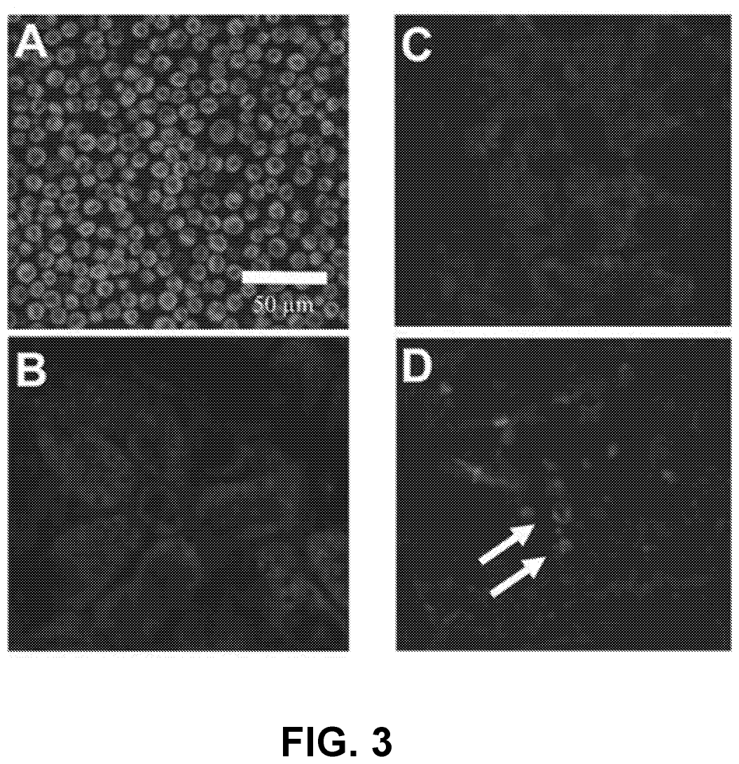

To determine whether transplanted PMVECs home to the post-ischemic kidney, cells were labeled with Celltracker red (CMTPX) just before administration and examined immediately following tissue harvest by confocal microscopy (FIG. 3A). There was no evidence of fluorescently labeled cells in post-ischemic kidneys at either 2 or 48 h following reperfusion (FIG. 3B and FIG. 3C). In contrast, some fluorescently labeled cells were readily apparent in the spleen (white arrows, FIG. 3D) and lung (not shown).

### Example 5: Human Endothelial Colony-Forming Cells-Conditioned Medium (ECFC-CM) Protects Against Renal I/R Injury

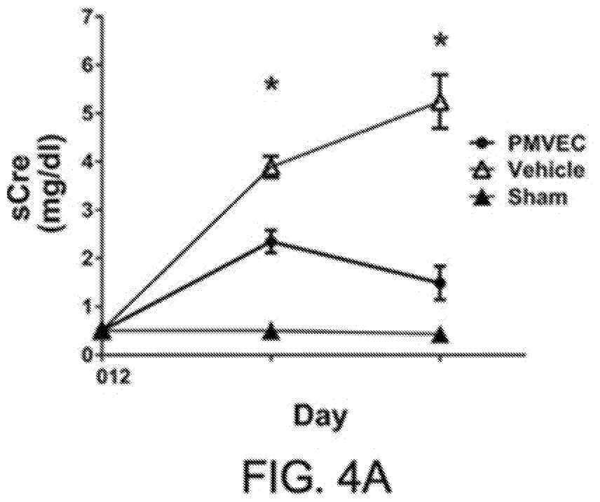

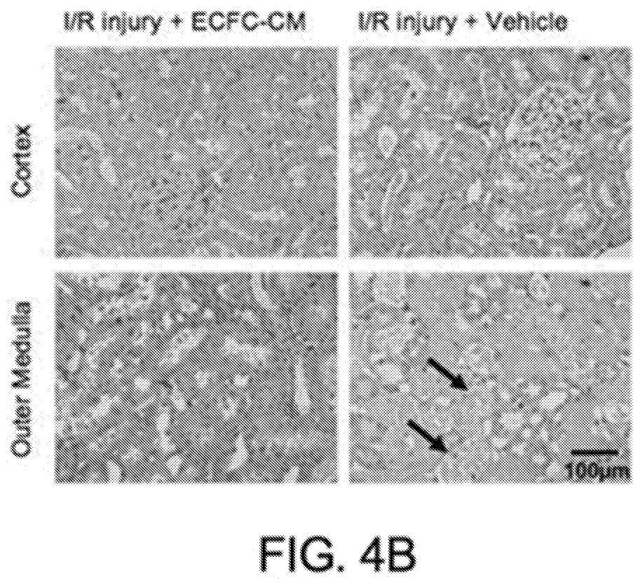

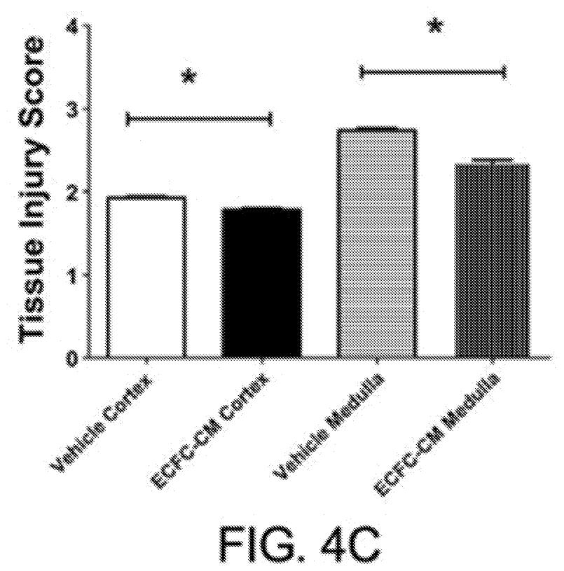

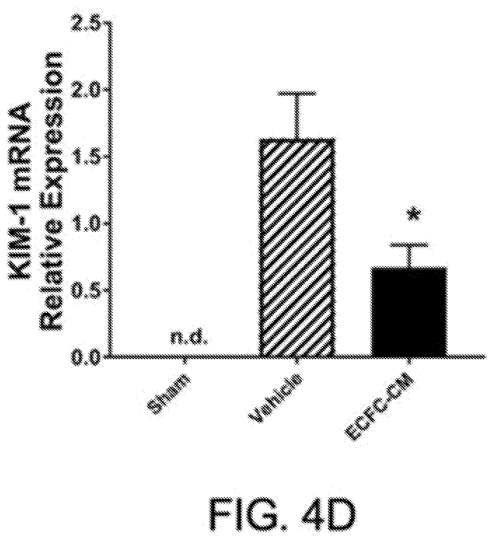

The lack of PMVECs homing indicates that soluble factors released from ECFCs may provide protection against impaired renal blood flow following renal I/R. Pilot studies were conducted to investigate whether soluble factors present in conditioned media of PMVEC may mediate protection from I/R injury. In one pilot study (n=4), 5 ml of PMVEC-CM was administered intraperitoneally. The increase in serum creatinine in PMVEC-CM-treated animals, measured 24 h following reperfusion, was significantly reduced by 44±10% relative to mock CM-treated post-I/R rats (data not shown). However, to increase the translational relevance of this research, we sought to utilize CM from human cord blood ECFCs, which have very high proliferative potential (Yoder et al. Blood 82: 385-391, 1993). In addition, we further modified our approach by concentrating hECFC-CM to facilitate a reasonable volume for intravascular administration. Relative to vehicle-injected control rats, hECFC-CM-treated rats manifested a significantly lower peak creatinine level following reperfusion (FIG. 4A). In addition, the level of histological damage was significantly less severe in ECFC-CM-treated rats compared with vehicle-treated animals at 2 days post-I/R (black arrows; FIG. 4B and FIG. 4C). To further assess renal injury, we evaluated KIM-1 mRNA expression and demonstrated that the expression of this marker for tubular injury was significantly reduced compared with vehicle-injected control rats (FIG. 4D).

### Example 6: Human ECFC-CM Preserves Medullary Blood Flow in the Early Post-Ischemic Period

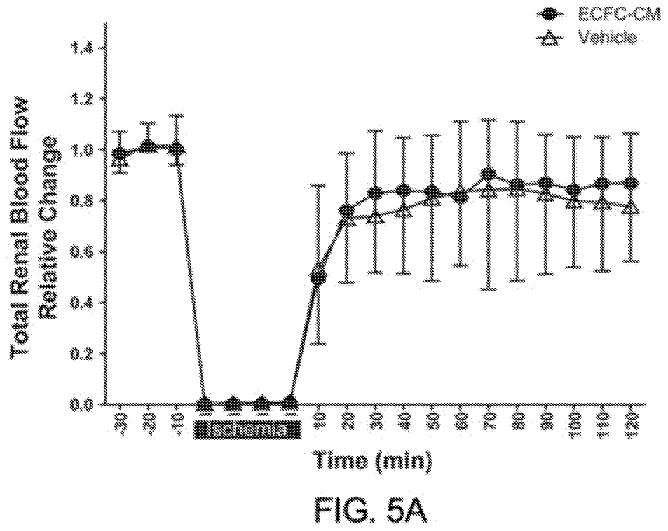

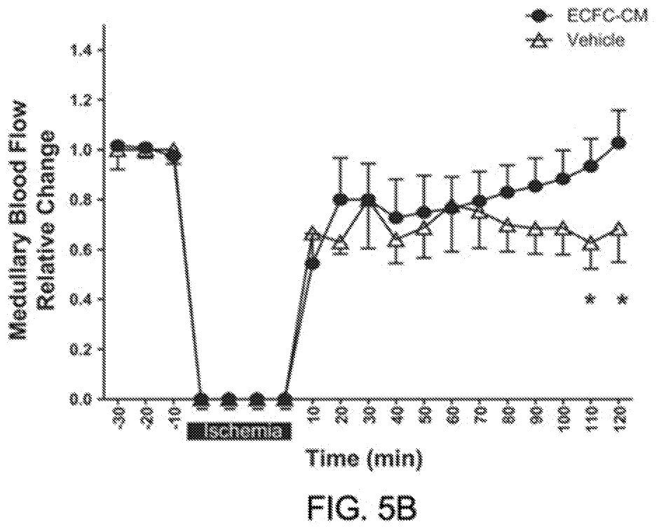

To determine whether human ECFC-CM administration preserves hemodynamic function post-ischemia, total RBF and outer MBF were measured. Similar to studies described in Example 3 (FIG. 2A), total RBF values recovered to ˜85% of control during the reperfusion phase and were not different between vehicle- and ECFC-CM-treated groups (FIG. 5A). In addition, MBF values returned toward control levels in hECFC-CM-treated animals but remained significantly suppressed below baseline in vehicle-treated controls (FIG. 5B).

### Example 7: Human ECFC-CM Reduces Adhesion Molecular Expression Following Recovery from I/R Injury

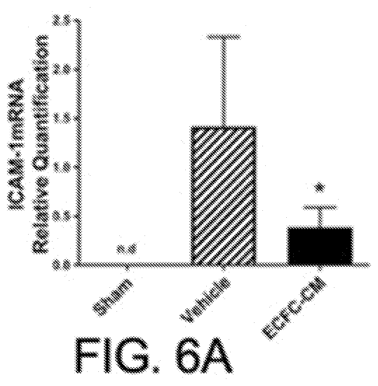

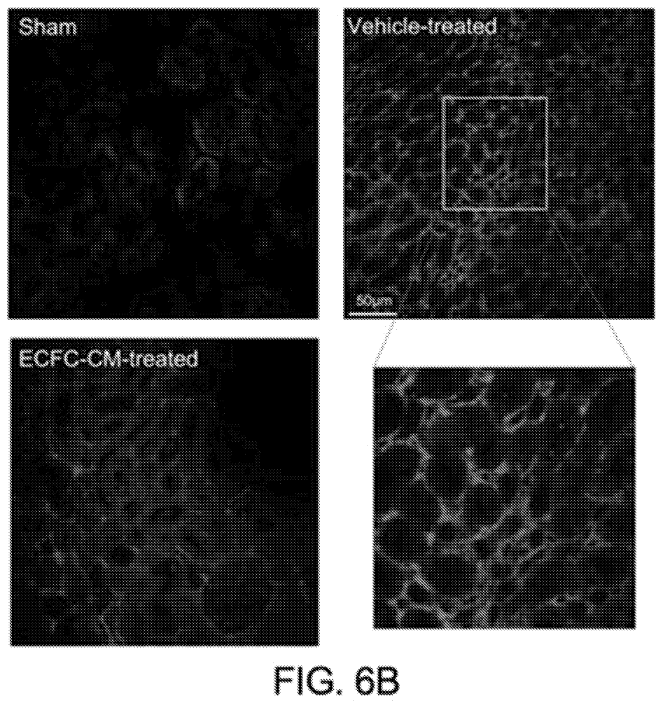

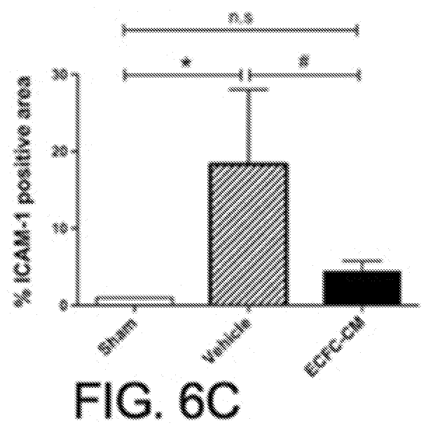

Previous data indicate that endothelial cell dysfunction leads to increased leukocyte adhesion, which may contribute to the severity of renal damage in the post-ischemic state (Basile et al., Kidney Int 66: 496-499, 2004). To determine whether hECFC-CM suppresses post-ischemic endothelial leukocyte adhesion, the mRNA expression of ICAM-1 was measured. ICAM-1 is an adhesion molecule known to be induced in endothelial cells in the early post-ischemic period. ICAM-1 mRNA expression was significantly increased within 5 h of reperfusion relative to sham (FIG. 6A). Similarly, ICAM-1 protein was not detectable in kidneys of sham-operated rats while it was prominently induced in peritubular capillaries of post-ischemic rats as indicated by immunofluorescence (FIG. 6B and FIG. 6C). Interestingly, both the mRNA expression of ICAM-1 (FIG. 6A) and the peritubular capillary protein expression of ICAM-1 (FIG. 6B and FIG. 6C) were significantly attenuated by infusion of hECFC-CM.

### Example 8: Human ECFC-CM Reduces Infiltration of Inflammatory Cells in Kidneys Following I/R

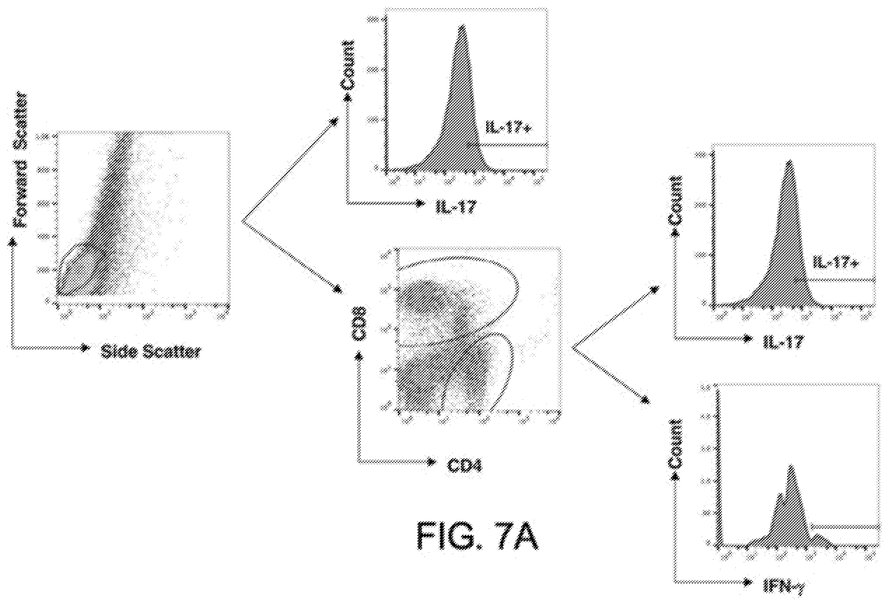

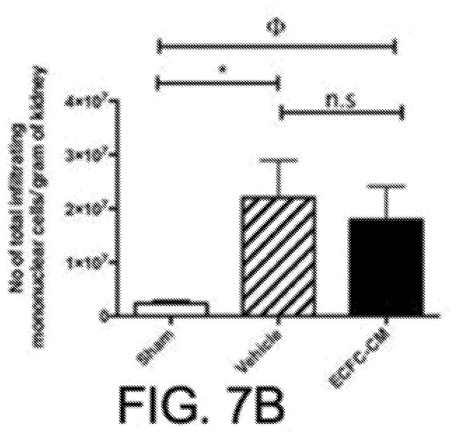

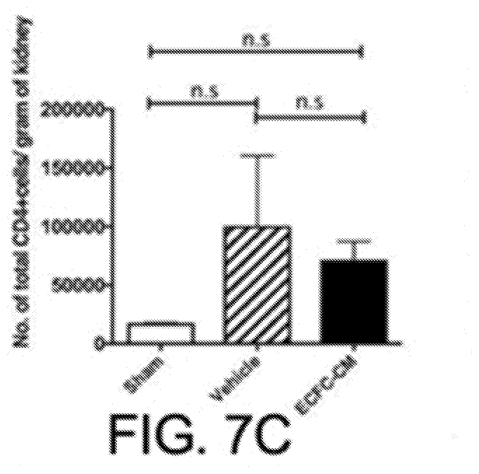

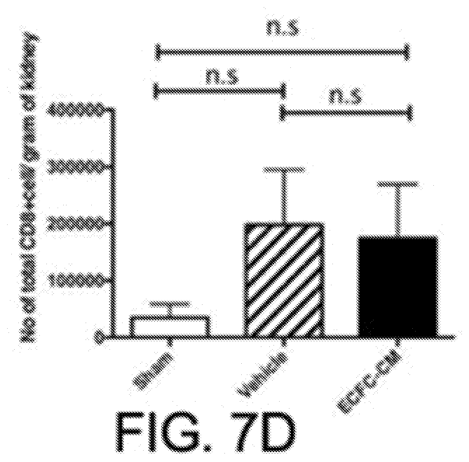

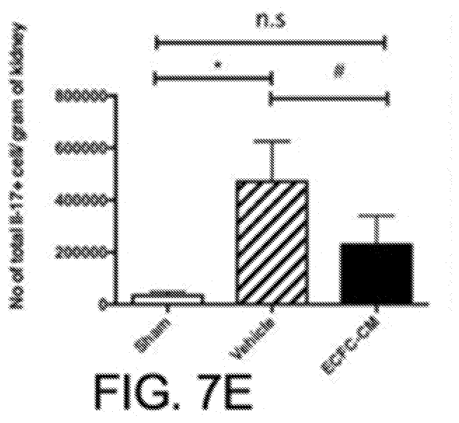

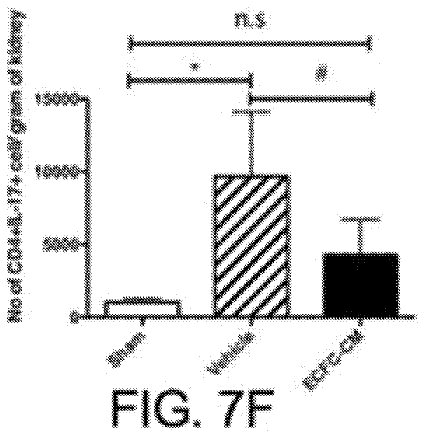

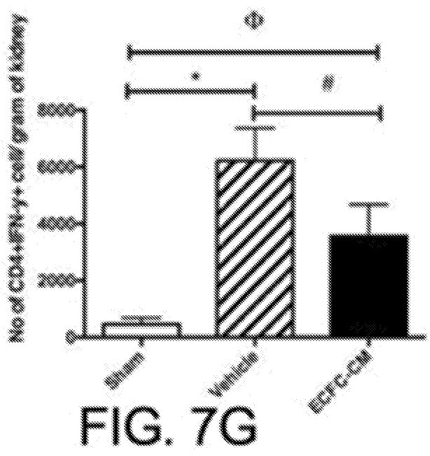

To determine whether hECFC-CM reduces post-ischemic inflammation, total and specific leukocyte populations were measured by fluorescence-activated cell sorting (FACS) following 2 days of recovery from renal I/R (FIG. 7A). The total number of leukocytes, as well as the total number of CD4+ and CD8+ cells, were significantly elevated following renal I/R, but these were not influenced by the hECFC-CM (FIGS. 7B-7D). Alterations in specific populations were observed. For example, the total number of cells expressing the cytokine IL-17 (FIG. 7E) as well as T-helper 17 cells (i.e., CD4+/IL17+) was significantly attenuated in hECFC-CM-treated rats (FIG. 7F). Moreover, Th-1 cells, defined as CD4+/IFN-γ+, were also significantly attenuated in hECFC-CM-treated rats (FIG. 7G). These data demonstrate that reductions in specific anti-inflammatory cells may contribute to ECFC-mediated protection from I/R-induced AKI.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### REFERENCES

1. Akcay A, Nguyen Q, Edelstein C L. Mediators of inflammation in acute kidney injury. Mediators Inflamm 2009: 1, 2009.
2. Alvarado-Moreno J A, Hernandez-Lopez R, Chavez-Gonzalez A, Yo- der M C, Rangel-Corona R, Isordia-Salas I, Hernandez-Juarez J, Cerbulo-Vazquez A, Gonzalez-Jimenez M A, Majluf-Cruz A. Endothelial colony-forming cells: Biological and functional abnormalities in patients with recurrent, unprovoked venous thromboembolic disease. Thromb Res 137: 157-168, 2016.
3. Alvarez D F, Huang L, King J A, ElZarrad M K, Yoder M C, Stevens T. Lung microvascular endothelium is enriched with progenitor cells that exhibit vasculogenic capacity. Am J Physiol Lung Cell Mol Physiol 294: L419-L430, 2007.
4. Asahara T, Masuda H, Takahashi T, Kalka C, Pastore C, Silver M, Kearne M, Magner M, Isner J M. Bone marrow origin of endothelial progenitor cells responsible for postnatal vasculogenesis in physiological and pathological neovascularization. Circ Res 85: 221-228, 1999.
5. Basile D P, Yoder M C. Renal endothelial dysfunction in acute kidney ischemia reperfusion injury. Cardiovasc Hematol Disord Drug Targets 14: 3-14, 2014.
6. Basile D P. The endothelial cell in ischemic acute kidney injury: implications for acute and chronic function. Kidney Int 72: 151-156, 2007.
7 Basile D P, Donohoe D, Roethe K, Osborn J L. Renal ischemic injury results in permanent damage to peritubular capillaries and influences long-term function. Am J Physiol Renal Physiol 281: F887-F899, 2001.
8. Basile D P, Dwinell M R, Wang S J, Shames B D, Donohoe D L, Chen S, Sreedharan R, Van Why S K. Chromosome substitution modulates resistance to ischemia reperfusion injury in Brown Norway rats. Kidney Int 83: 242-250, 2013.
9. Basile D P, Friedrich J L, Spahic J, Knipe N, Mang H, Leonard E C, Changizi-Ashtiyani S, Bacallao R L, Molitoris B A, Sutton T A. Impaired endothelial proliferation and mesenchymal transition contribute to vascular rarefaction following acute kidney injury. Am J Physiol Renal Physiol 300: F721-F733, 2011.
10. Basile D P, Zeng P, Friedrich J L, Leonard E C, Yoder M C. Low proliferative potential and impaired angiogenesis of cultured rat kidney endothelial cells. Microcirculation 19: 598-609, 2012.
11. Bhardwaj G, Murdoch B, Wu D, Baker D P, Williams K P, Chadwick K, Ling L E, Karanu F N, Bhatia M. Sonic hedgehog induces the proliferation of primitive human hematopoietic cells via BMP regulation. Nat Immunol. (2001) 2(2):172-80.
12. Bhatia et al. Bone morphogenetic proteins regulate the developmental program of human hematopoietic stem cells. J Exp Med. (1999) 189(7):1139-48.
13. Boesen E I, Crislip G R, Sullivan J C. Use of ultrasound to assess renal reperfusion and P-selectin expression following unilateral renal ischemia. Am J Physiol Renal Physiol 303: F1333-F1340, 2012.
14. Brezis M, Rosen S. Hypoxia of the renal medulla—its implications for disease. N Engl J Med 332: 647-655, 1995.
15. Brodsky S V, Yamamoto T, Tada T, Kim B, Chen J, Kajiya F, Goligorsky M S. Endothelial dysfunction in ischemic acute renal failure: rescue by transplanted endothelial cells. Am J Physiol Renal Physiol 282: F1140-F1149, 2002.
16. Burger D, Villas J L, Akbari S, Dehak H, Knoll W, Gutsol A, Carter A, Touyz R M, Allan D S, Burns K D. Human endothelial colony-forming cells protect against acute kidney injury: role of exosomes. Am J Pathol 185: 2309-2323, 2015.
17. Burgos-Silva M, Semedo-Kuriki P, Donizetti-Oliveira C, Costa P B, Cenedeze M A, Hiyane M I, Pacheco-Silva A, Camara N O S. Adipose tissue-derived stem cells reduce acute and chronic kidney damage in mice. PLoS One 10: e0142183, 2015.
18. Cantaluppi V, Gatti S, Medica D, Figliolini F, Bruno S, Deregibus M C, Sordi A, Biancone L, Tetta C, Camussi G. Microvesicles derived from endothelial progenitor cells protect the kidney from ischemia-reperfusion injury by microRNA-dependent reprogramming of resident renal cells. Kidney Int 82: 412-427, 2012.
19. Chadwick K, Wang L, Li L, Menendez P, Murdoch B, Rouleau A, Bhatia M. Cytokines and BMP-4 promote hematopoietic differentiation of human embryonic stem cells. Blood. (2003) 102(3):906-15.
20. Chan A J, Alikhan M A, Odobasic D, Gan P Y, Khouri M B, Steinmetz O M, Mansell A S, Kitching A R, Holdsworth S R, Summers S A. Innate IL-17A-producing leukocytes promote acute kidney injury via inflammasome and Toll-like receptor activation. Am J Pathol 184: 1411-1418, 2014.
21. Choi K D, Yu J, Smuga-Otto K, Salvagiotto G, Rehrauer W, Vodyanik M, Thomson J, Slukvin I. Hematopoietic and endothelial differentiation of human induced pluripotent stem cells. Stem Cells. (2009) 27(3):559-67.
22. Cimato T, Beers J, Ding S, Ma M, McCoy J P, Boehm M, Nabel E G. Neuropilin-1 identifies endothelial precursors in human and murine embryonic stem cells before CD34 expression. Circulation. 2009 Apr. 28; 119(16):2170-8.
23. Collett J A, Mehrotra P, Crone A, Merfeld-Clauss S, March K L, Basile D P. Human adipose stromal cell therapy improves survival and reduces renal inflammation and capillary rarefaction in acute kidney injury. J Cell Mol Med 2017.
24. Conesa E L, Valero F, Nadal J C, Fenoy F J, Lopez B, Arregui B, Salom M G. N-acetyl-L-cysteine improves renal medullary hypoperfusion in acute renal failure. Am J Physiol Regul Integr Comp Physiol 281: R730-R737, 2001.
25. Davidson A J, Zon L I. Turning mesoderm into blood: the formation of hematopoietic stem cells during embryogenesis. Curr Top Dev Biol. (2000) 50:45-60.
26. Day Y J, Huang L, Ye H, Li L, Linden J, Okusa M D. Renal ischemia-reperfusion injury and adenosine 2A receptor-mediated tissue protection: the role of CD4+ T cells and IFN-gamma. J Immunol 176: 3108-3114, 2006.
27. de Braganca A C, Volpini R A, Mehrotra P, Andrade L, Basile D P. Vitamin D deficiency contributes to vascular damage in sustained ischemic acute kidney injury. Physiol Rep 4: e12829, 2016.
28. Friedewald J J, Rabb H. Inflammatory cells in ischemic acute renal failure. Kidney Int 66: 486-491, 2004.
29. Fuller T F, Sattler B, Binder L, Vetterlein F, Ringe B, Lorf T. Reduction of severe ischemia/reperfusion injury in rat kidney grafts by a soluble P-selectin glycoprotein ligand. Transplantation 72: 216-222, 2001.
30. Haller H, Dragun D, Miethke A, Park J K, Weis A, Lippoldt A, Gross V, Luft F C. Antisense oligonucleotides for ICAM-1 attenuate reperfusion injury and renal failure in the rat. Kidney Int 50: 473-480, 1996.
31. Herter J M, Rossaint J, Spieker T, Zarbock A. Adhesion molecules involved in neutrophil recruitment during sepsis-induced acute kidney injury. J Innate Immun 6: 597-606, 2014.
32. Huber T L, Zhou Y, Mead P E, Zon L I. Cooperative effects of growth factors involved in the induction of hematopoietic mesoderm. Blood. (1998) 92(11):4128-37
33. Kelly K J, Williams W W Jr, Colvin R B, Bonventre J V. Antibody to intercellular adhesion molecule 1 protects the kidney against ischemic injury. Proc Natl Acad Sci USA 91: 812-816, 1994.
34. Kim M, Ham A, Kim J Y, Brown K M, D′Agati V D, Lee H T. The volatile anesthetic isoflurane induces ecto-5=-nucleotidase (CD73) to protect against renal ischemia and reperfusion injury. Kidney Int 84: 90-103, 2013.
35. Lee S, Huen S, Nishio H, Nishio S, Lee H K, Choi B-S, Ruhrberg C, Cantley L G. Distinct macrophage phenotypes contribute to kidney injury and repair. J Am Soc Nephrol 22: 317-326, 2011.
36. Li B, Cohen A, Hudson T E, Motlagh D, Amrani D L, Duffield J S. Mobilized human hematopoietic stem/progenitor cells promote kidney repair after ischemia/reperfusion injury. Circulation 121: 2211-2220, 2010.
37. Livak K J, Schmittgen T D. Analysis of relative gene expression data using real-time quantitative PCR and the 2(-Delta C(T)) Method. Methods 25: 402-408, 2001.
38. Maherali N, Sridharan R, Xie W, Utikal J, Eminli S, Arnold K, Stadtfeld M, Yachechko R, Tchieu J, Jaenisch R, Plath K, Hochedlinger K. Directly reprogrammed fibroblasts show global epigenetic remodeling and widespread tissue contribution. Cell Stem Cell (2007) 1, 55-70.
39. Marshall C J, Kinnon C, Thrasher A J. Polarized expression of bone morphogenetic protein-4 in the human aorta-gonad-mesonephros region. Blood. (2000); 96(4):1591-3.
40. Mcniece I K, Bertoncello I, Kriegler A B, Quesenberry P J. Colony-forming cells with high proliferative potential (HPP-CFC). Int J Cell Cloning 8: 146-160, 1990.
41. Mehrotra P, Patel J B, Ivancic C M, Collett J A, Basile D P. Th-17 cell activation in response to high salt following acute kidney injury is associated with progressive fibrosis and attenuated by AT-1R antagonism. Kidney Int 88: 776-784, 2015.
42. Meissner A, Wernig M, Jaenisch R. Direct reprogramming of genetically unmodified fibroblasts into pluripotent stem cells. Nat Biotechnol. (2007) 25(10):1177-81.
43. Molitoris B A. Therapeutic translation in acute kidney injury: the epithelial/endothelial axis. J Clin Invest 124: 2355-2363, 2014.
44. Molitoris B A, Sandoval R, Sutton T A. Endothelial injury and dysfunction in ischemic acute renal failure. Crit Care Med 30, Suppl: S235-S240, 2002.
45. Molitoris B A, Sutton T A. Endothelial injury and dysfunction: role in the extension phase of acute renal failure. Kidney Int 66: 496-499, 2004.
46. Morigi M, Introna M, Imberti B, Coma D, Abbate M, Rota C, Rottoli D, Benigni A, Perico N, Zoja C, Rambaldi A, Remuzzi A, Remuzzi G. Human bone marrow mesenchymal stem cells accelerate recovery of acute renal injury and prolong survival in mice. Stem Cells 26: 2075-2082, 2008.
47. Naito H, Kidoya H, Sakimoto S, Wakabayashi T, Takakura N. Identification and characterization of a resident vascular stem/progenitor cell population in preexisting blood vessels. EMBO J 31: 842-855, 2012.
48. Nakagawa M1, Koyanagi M, Tanabe K, Takahashi K, Ichisaka T, Aoi T, Okita K, Mochiduki Y, Takizawa N, Yamanaka S. Generation of induced pluripotent stem cells without Myc from mouse and human fibroblasts. Nature Biotechnol. (2007) 26, 101-106.
49. Nguyen H, Chiasson V L, Chatterjee P, Kopriva S E, Young K J, Mitchell B M. Interleukin-17 causes Rho-kinase-mediated endothelial dysfunction and hypertension. Cardiovasc Res 97: 696-704, 2013.
50. Okita K, Ichisaka T, Yamanaka S. Generation of germline-competent induced pluripotent stem cells. Nature (2007) 448, 313-317.
51. Patschan D, Krupincza K, Patschan S, Zhang Z, Hamby C, Goligorsky M S. Dynamics of mobilization and homing of endothelial progenitor cells after acute renal ischemia: modulation by ischemic preconditioning. Am J Physiol Renal Physiol 291: F176-F185, 2006.
52. Phillips S A, Pechman K R, Leonard E C, Friedrich J L, Bian J-T, Beal A G, Basile D P. Increased ANG II sensitivity following recovery from acute kidney injury: role of oxidant stress in skeletal muscle resistance arteries. Am J Physiol Regul Integr Comp Physiol 298: R1682-R1691, 2010.
53. Rundback et al. Key Concepts in Critical Limb Ischemia: Selected Proceedings from the 2015 Vascular Interventional Advances Meeting. Ann Vasc Surg. (2017) 38:191-205.
54. Rabb H, Mendiola C C, Saba S R, Dietz J R, Smith C W, Bonventre J V, Ramirez G. Antibodies to ICAM-1 protect kidneys in severe ischemic reperfusion injury. Biochem Biophys Res Commun 211: 67-73, 1995.
55. Rajashekhar G, Traktuev D O, Roell W C, Johnstone B H, Merfeld-Clauss S, Van Natta B, Rosen E D, March K L, Clauss M. IFATS collection: Adipose stromal cell differentiation is reduced by endothelial cell contact and paracrine communication: role of canonical Wnt signaling. Stem Cells 26: 2674-2681, 2008.
56. Regner K R, Roman R J. Role of medullary blood flow in the pathogenesis of renal ischemia-reperfusion injury. Curr Opin Nephrol Hypertens 21: 33-38, 2012.
57. Regner K R, Zuk A, Van Why S K, Shames B D, Ryan R P, Falck J R, Manthati V L, McMullen M E, Ledbetter S R, Roman R J. Protective effect of 20-HETE analogues in experimental renal ischemia reperfusion injury. Kidney Int 75: 511-517, 2009.
58. Rehman J, Li J, Orschell C M, March K L. Peripheral blood “endothelial progenitor cells” are derived from monocyte/macrophages and secrete angiogenic growth factors. Circulation 107: 1164-1169, 2003.
59. Rehman J, Traktuev D, Li J, Merfeld-Clauss S, Temm-Grove C J, Bovenkerk J E, Pell C L, Johnstone B H, Considine R V, March K L. Secretion of angiogenic and antiapoptotic factors by human adipose stromal cells. Circulation 109: 1292-1298, 2004.
60. Shih Y C, Lee P Y, Cheng H, Tsai C H, Ma H, Tarng D C. Adipose-derived stem cells exhibit antioxidative and antiapoptotic properties to rescue ischemic acute kidney injury in rats. Plast Reconstr Surg 132: 940e-951e, 2013.
61. Singbartl K, Forlow S B, Ley K. Platelet, but not endothelial, P-selectin is critical for neutrophil-mediated acute postischemic renal failure. FASEB J 15: 2337-2344, 2001.
62. Singbartl K, Green S A, Ley K. Blocking P-selectin protects from ischemia/reperfusion-induced acute renal failure. FASEB J 14: 48-54, 2000.
63. Samuel et al. Generation of functionally competent and durable engineered blood vessels from human induced pluripotent stem cells. Proc Natl Acad Sci U S A 2013 Jul. 30; 110(31):12774-9.
64. Takahashi, K. & Yamanaka, S. Induction of pluripotent stem cells from mouse embryonic and adult fibroblast cultures by defined factors. Cell (2006) 126(4):663-76.
65. Togel F, Cohen A, Zhang P, Yang Y, Hu Z, Westenfelder C. Autologous and allogeneic marrow stromal cells are safe and effective for the treatment of acute kidney injury. Stem Cells Dev 18: 475-486, 2009.
66. Togel F, Westenfelder C. The role of multipotent marrow stromal cells (MSCs) in tissue regeneration. Organogenesis 7: 96-100, 2011.
67. Togel F, Yang Y, Zhang P, Hu Z, Westenfelder C. Bioluminescence imaging to monitor the in vivo distribution of administered mesenchymal stem cells in acute kidney injury. Am J Physiol Renal Physiol 295: F315-F321, 2008.
68. Togel F, Zhang P, Hu Z, Westenfelder C. VEGF is a mediator of the renoprotective effects of multipotent marrow stromal cells in acute kidney injury. J Cell Mol Med 13, 8B: 2109-2114, 2009.
69. Tögel F E, Westenfelder C. Kidney protection and regeneration following acute injury: progress through stem cell therapy. Am J Kidney Dis 60: 1012-1022, 2012.
70. Togel F E, Westenfelder C. Mesenchymal stem cells: a new therapeutic tool for AKI. Nat Rev Nephrol 6: 179-183, 2010.
71. Varu et al. Critical limb ischemia. (2010) Journal of Vascular Surgery 51(1): 230-41.
72. Vella F. Textbook of Clinical Chemistry. Tietz N W, Editor. Philadelphia, Pa.: Saunders, 1986.
73. Villanueva S, Carreño J E, Salazar L, Vergara C, Strodthoff R, Fajre F, Céspedes C, Sáez P J, Irarrázabal C, Bartolucci J, Figueroa F, Vio C P. Human mesenchymal stem cells derived from adipose tissue reduce functional and tissue damage in a rat model of chronic renal failure. Clin Sci (Lond) 125: 199 —210, 2013.
74. Viñas J L, Burger D, Zimpelmann J, Haneef R, Knoll W, Campbell P, Gutsol A, Carter A, Allan D S, Burns K D. Transfer of microRNA-486-5p from human endothelial colony-forming cell-derived exosomes reduces ischemic kidney injury. Kidney Int 90: 1238-1250, 2016.
75. Wernig M, Meissner A, Foreman R, Brambrink T, Ku M, Hochedlinger K, Bernstein B E, Jaenisch R. In vitro reprogramming of fibroblasts into a pluripotent ES-cell-like state. Nature (2007) 448, 318-324.
76. Wernig M, Meissner A, Cassady J P, Jaenisch R. c-Myc is dispensable for direct reprogramming of mouse fibroblasts. Cell Stem Cell. (2008) 10; 2(1):10-2.
77. Westenfelder C, Togel F E. Protective actions of administered mesenchymal stem cells in acute kidney injury: relevance to clinical trials. Kidney Int Suppl 1, Suppl: 103-106, 2011.
78. Yao W, Hu Q, Ma Y, Xiong W, Wu T, Cao J, Wu D. Human adipose-derived mesenchymal stem cells repair cisplatin-induced acute kidney injury through antiapoptotic pathways. Exp Ther Med 10: 468-476, 2015.
79. Yoder M C. Is endothelium the origin of endothelial progenitor cells? Arterioscler Thromb Vasc Biol 30: 1094-1103, 2010.
80. Yoder M C, Du X X, Williams D A. High proliferative potential colony-forming cell heterogeneity identified using counterflow centrifugal elutriation. Blood 82: 385-391, 1993.
81. Yoder M C, Mead L E, Prater D, Krier T R, Mroueh K N, Li F, Krasich R, Temm C J, Prchal J T, Ingram D A. Redefining endothelial progenitor cells via clonal analysis and hematopoietic stem/progenitor cell principals. Blood 109: 1801-1809, 2007.
82. Yokota N, Daniels F, Crosson J, Rabb H. Protective effect of T cell depletion in murine renal ischemia-reperfusion injury. Transplantation 74: 759-763, 2002.
83. Ysebaert D K, De Greef K E, De Beuf A, Van Rompay A R, Vercauteren S, Persy V P, De Broe M E. T cells as mediators in renal ischemia/reperfusion injury. Kidney Int 66: 491-496, 2004.
84. Ysebaert D K, De Greef K E, Vercauteren S R, Ghielli M, Verpooten G A, Eyskens E J, De Broe M E. Identification and kinetics of leukocytes after severe ischaemia/reperfusion renal injury. Nephrol Dial Transplant 15: 1562-1574, 2000.
85. Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science (2007) 318, 1917-1920.
86. Zander A R, Lange C, Westenfelder C. Mesenchymal stromal cells: main factor or helper in regenerative medicine? Kidney Int Suppl 1: 74 —76, 2011.
87. Zhang Y, Fisher N, Newey S E, Smythe J, Tatton L, Tsaknakis G, Forde S P, Carpenter L, Athanassopoulos T, Hale S J, Ferguson D J, Tyler M P, Watt S M. The impact of proliferative potential of umbilical cord-derived endothelial progenitor cells and hypoxia on vascular tubule formation in vitro. Stem Cells Dev 18: 359-376, 2009.
88. Zizzi H C, Zibari G B, Granger D N, Singh I, Cruz L D, Abreo F, McDonald J C, Brown M F. Quantification of P-selectin expression after renal ischemia and reperfusion. J Pediatr Surg 32: 1010-1013, 1997.

What is claimed is:

1. A serum-free composition comprising a chemically defined medium conditioned by endothelial colony-forming cells (ECFCs).

2. A composition of claim 1, wherein the endothelial colony-forming cells are derived from multipotent stem cells or are derived from pluripotent stem cells.

3. The composition of claim 2, wherein the multipotent stem cells are cord blood stem cells.

4. The composition of claim 2, wherein the ECFCs:
a. are derived from pluripotent stem cells without co-culture with bone marrow cells;
b. are derived from pluripotent stem cells without embryoid body formation; or
c. do not express α-smooth muscle actin (α-SMA)

5. A kit comprising the composition of claim 1.

6. The serum-free composition of claim 4 wherein about 35-95% of the ECFCs are HPP-ECFCs and capable of self-renewal.

7. The serum-free composition of claim 4 wherein the ECFCs form blood vessels when implanted in vivo in a mammal.
